(19) United States
(12) Patent Application Publication
(10) Pub. No.: US 2002/0098495 A1
(43) Pub. Date: Jul. 25, 2002

(54) Proteins associated with aging

(75) Inventors: Glenna Burmer, Seattle, WA (US); David Pritchard, Seattle, WA (US); Joseph P. Brown, Seattle, WA (US); Vasiliki Demas, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LifeSpan BioSciences, Inc., Seattle, WA (US)

(21) Appl. No.: 09/898,730

(22) Filed: Jul. 3, 2001

Related U.S. Application Data
(60) Provisional application No. 60/216,470, filed on Jul. 6, 2000.

(51) Int. Cl. C12Q 1/68; G01N 33/53; G01N 33/567

(52) U.S. Cl. 435/6; 435/7.1; 435/7.2

(57) ABSTRACT

This invention relates to the discovery of nucleic acids and proteins associated with the aging processes, such as cell proliferation and senescence. The identification of these aging-associated nucleic acids and proteins have diagnostic uses in detecting the aging status of a cell population as well as applications for gene therapy and the delaying of the aging process.

### CROSS-REFERENCES TO RELATED APPLICATIONS

The present application claims priority to U.S. Ser. No. 60/216,470, filed Jul. 6, 2000, herein incorporated by reference in its entirely.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

Normal human diploid cells have a finite potential for proliferative growth (Hayflick et al., Exp. Cell Res. 25:585 (1961); Hayflick, Exp. Cell Res. 37:614 (1965)). Indeed, under controlled conditions, in vitro cultured human cells can maximally proliferate only to about 80 cumulative population doublings. The proliferative potential of such cells has been found to be a function of the number of cumulative population doublings which the cell has undergone (Hayflick et al. (1961), supra; Hayflick et al. (1965), supra). This potential is also inversely proportional to the in vivo age of the cell donor (Martin et al., Lab. Invest. 23:86 (1979); Goldstein et al., Proc. Natl. Acad. Sci. U.S.A. 64:155 (1969); Schneider, Proc. Natl. Acad. Sci. U.S.A. 73:3584 (1976); LeGuilty et al., Gereontologia 19:303 (1973)).

Cells that have exhausted their potential for proliferative growth are said to have undergone “senescence.” Cellular senescence in vitro is exhibited by morphological changes and is accompanied by the failure of a cell to respond to exogenous growth factors. Cellular senescence, thus, represents a loss of the proliferative potential of the cell. Although a variety of theories have been proposed to explain the phenomenon of cellular senescence in vitro, experimental evidence suggests that the age-dependent loss of proliferative potential may be the function of a genetic program (Orgel, Proc. Natl. Acad. Sci. U.S.A. 49:517 (1963); De Mars et al., Human Genet. 16:87 (1972); Buchwald, Mutat. Res. 44:401 (1977); Martin et al., Amer. J. Pathol. 74:137 (1974); Smith et al., Mech. Age. Dev. 13:387 (1980); and Kirkwood et al., Theor. Biol. 53:481 (1975)).

The prospect of reversing senescence and restoring the proliferative potential of cells has implications in many fields of endeavor. Many of the diseases of old age are associated with the loss of this potential. Moreover, the tragic disease, progeria, which is characterized by accelerated aging, is associated with the loss of proliferative potential of cells. Werner Syndrome and Hutchinson-Gilford Syndrome are two forms of progeria. A major clinical difference between the two is that the onset of Hutchinson-Gilford Syndrome (sometimes called progeria of childhood) occurs within the first decade of life, whereas the first evidence of Werner Syndrome (sometimes called progeria of adulthood) appears only after puberty, with the full symptoms becoming manifest in individuals 20 to 30 years old.

More particularly, Hutchinson-Gilford syndrome is a very rare progressive disorder of childhood characterized by premature aging (progeria), growth delays occurring in the first year of life resulting in short stature and low weight, deterioration of the layer of fat beneath the skin (subcutaneous adipose tissue), and characteristic craniofacial abnormalities, including an abnormally small face, underdeveloped jaw (micrognathia), unusually prominent eyes and/or a small, “beak-like” nose. In addition, during the first year or two of life, scalp hair, eyebrows and eyelashes may become sparse, and veins of the scalp may become unusually prominent. Additional symptoms and physical findings may include joint stiffness, repeated nonhealing fractures, a progressive aged appearance of the skin, delays in tooth eruption (dentition) and/or malformation and crowding of the teeth. Individuals with the disorder typically have normal intelligence. In most cases, affected individuals experience premature, widespread thickening and loss of elasticity of artery walls (arteriosclerosis), potentially resulting in life-threatening complications. Hutchinson-Gilford Progeria Syndrome is thought to be due to an autosomal dominant genetic change (mutation) that occurs for unknown reasons (sporadic).

Moreover, Werner Syndrome patients prematurely develop many age related diseases, including arteriosclerosis, malignant neoplasma, type II diabetes, osteoporosis, ocular cataracts, early graying, loss of hair, skin atrophy and aged appearance. Although Werner Syndrome patients prematurely show some of the signs of aging (such as graying of the hair and hair loss, atherosclerosis, osteoporosis and type II diabetes mellitus), they fail to show others. For example, they exhibit no premature cognitive decline or Alzheimer's symptoms. In addition, they experience many symptoms not associated with normal aging (such as ulceration of the skin, particularly around the ankles, alteration of the vocal chords resulting in a high-pitched voice, and an absence of the growth spurt that normally occurs after puberty).

In view of the devastating effects of the aging process and age-related diseases, reversing senescence and restoring the proliferative potential of cells would have far-reaching implications for the treatment of these diseases, of other age-related disorders, and, of aging per se. In addition, the restoration of proliferative potential of cultured cells has uses in medicine and in the pharmaceutical industry. The ability to immortalize non-transformed cells can be used to generate an endless supply of certain tissues and also of cellular products.

### SUMMARY OF THE INVENTION

The present invention provides isolated nucleic acids and proteins associated with aging, as well as aging-related diseases (e.g., Werner Syndrome, Progeria, skin cancer, etc.). Such sequences can be used to determine the aging status of a cell population, e.g., whether a cell is aging or is undergoing senescence. Moreover, the present invention provides sequences indicative of the proliferation state or youth of a cell. Such sequences can also be targeted and their level of expression altered by, for example, gene therapy methods (e.g., by altering the subject sequences). Such methods can be used, for example, to slow or stop the aging process of the cell population, to arrest the growth of a proliferating cell population, such as a tumor cell population, to promote division in cells which are prematurely arrested, to determine that a cell population is healthy and rapidly dividing, and to determine that a cell population is not dividing and proliferating.

In one aspect, the present invention provides a method for detecting whether a tissue is undergoing senescence, the method comprising the step of detecting the overexpression or the underexpression of a senescence-associated molecule of interest according to Table 1 in a cell or tissue, wherein overexpression or underexpression of the molecule is indicative of senescence. In some embodiments overexpression of the molecule is indicative of senescence, and the molecule is overexpressed in the cell or tissue. In other embodiments, underexpression of the molecule is indicative of senescence, and the molecule is underexpressed in the cell or tissue. The molecule detected can be an mRNA encoding a senescence-associated protein. Alternatively, a senescence-associated protein can also be detected using an immunoassay.

The present invention also provides a method for identifying a modulator of cellular aging, the method comprising the steps of culturing a cell in the presence of the modulator to form a first cell culture; contacting RNA or cDNA from the first cell culture with a probe which comprises a polynucleotide sequence that encodes a protein associated with aging; determining whether the amount of probe which hybridizes to the RNA or cDNA from the first cell culture is increased or decreased relative to the amount of probe which hybridizes to RNA or cDNA from a second cell culture grown in the absence of the modulator; and detecting the presence or absence of an increased proliferative potential, or of altered aging properties, in the first cell culture relative to the second cell culture. Altered aging properties may include, for example, a change in cellular morphology or a resumption of an aged cell's ability to respond to exogenous growth factors. In one embodiment, the polynucleotide sequences that encode proteins associated with aging are selected from the group consisting of the sequences set forth in Table 1. In a preferred embodiment, the first and second cell cultures are obtained from a fibroblast cell.

The present invention further provides a method for identifying a modulator of a young cell, the method comprising the steps of culturing a cell in the presence of the modulator to form a first cell culture; contacting RNA or cDNA from the first cell culture with a probe which comprises a polynucleotide sequence associated with young cells; determining whether the amount of probe which hybridizes to the RNA or cDNA from the first cell culture is increased or decreased relative to the amount of probe which hybridizes to RNA or cDNA from a second cell culture grown in the absence of the modulator; and detecting the presence or absence of altered aging properties in the first cell culture relative to the second cell culture. Altered aging properties include, for example, a change in cellular morphology; a change in the proliferative potential of a cell, wherein an aged cell regains proliferative potential, or a resumption of an aged cell's ability to respond to exogenous growth factors. In one embodiment, the polynucleotide sequences associated with young cells are selected from the group consisting of the sequences set forth in Table 1.

In another aspect, the present invention is directed to a method for inhibiting cell senescence, the method comprising the step of introducing into a cell a cell a senescence-associated molecule, wherein underexpression of the senescence-associated molecule is indicative of senescence. In one embodiment, the senescence-associated molecule introduced into the cell is a nucleic acid encoding a senescence-associated protein. In another embodiment, a senescence-associated protein is introduced into the cell. In one embodiment, the molecule associated with senescence is selected from the group consisting of the sequences set forth in Table 1.

In addition, the present invention also provides a method for inhibiting cell senescence, the method comprising the step of inhibiting in a cell a senescence-associated molecule, wherein overexpression of the senescence-associated molecule is indicative of senescence. In a preferred embodiment, the senescence-associated molecule is inhibited using an antisense polynucleotide. In another preferred embodiment, the senescence-associated molecule is inhibited using an antibody that specifically binds to the senescence-associated protein. In a preferred embodiment, the senescence-associated molecule is selected from the group consisting of the sequences set forth in Table 1.

In yet another aspect, the present invention provides a method for inhibiting cell senescence in a patient in need thereof, the method comprising the step of administering to the patient a compound that modulates the senescence of a cell.

The present invention is also directed to kits for detecting whether a cell is undergoing senescence, the kit comprising a probe which comprises a polynucleotide sequence associated with aging and a label for detecting the presence of the probe. In a preferred embodiment, the cell is a fibroblast cell. In one embodiment, the probe comprises at least 10 nucleotides from a polynucleotide sequence selected from the group of the sequences listed in Table 1. Additionally, the kit can further comprise a plurality of probes each of which comprises a polynucleotide sequence associated with aging, and a label or labels for detecting the presence of the plurality of probes. The probes can optionally be immobilized on a solid support (e.g., a chip).

### BRIEF DESCRIPTION OF THE DRAWINGS

Not applicable.

### DESCRIPTION OF THE SPECIFIC EMBODIMENTS

### I. INTRODUCTION

The present invention provides nucleic acids and proteins that are indicative of aging and/or of cell death (senescence) and cell proliferation. Host cells, vectors, and probes are described, as are antibodies to the proteins and uses of the proteins as antigens. The present invention provides methods for obtaining and expressing nucleic acids, methods for purifying gene products, other methods that can be used to detect and quantify the expression and quality of the gene product (e.g., proteins), and uses for both the nucleic acids and the gene products.

This invention relies on routine techniques in the field of recombinant genetics. A basic text disclosing the general methods of use in this invention is Sambrook et al., Molecular Cloning, A Laboratory Manual, Cold Spring Harbor Publish., Cold Spring Harbor, N.Y. 2nd ed. (1989); and Kriegler, Gene Transfer and Expression: A Laboratory Manual, Freeman, N.Y. (1990). Unless otherwise stated all enzymes are used in accordance with the manufacturer's instructions.

### II. DEFINITIONS

In the context of the present invention, “aging” of a cell or tissue encompasses the aging processes due to intrinsic aging, as well as disease- or extrinsic factors-related aging. “Aging” of a cell or tissue is characterized by, e.g., cell death (senescence) and loss of cell proliferation potential, as well as any of a number of characteristic structural and/or molecular features. In the context of the present invention, “aging” refers to all the stages of the process.

The terms “aging-associated” or “senescence-associated” refer to the relationship of a nucleic acid and its expression, or lack thereof, or a protein and its level or activity, or lack thereof, to the onset and/or progression of aging or senescence in a subject. For example, aging or senescence can be associated with expression of a particular gene that is not expressed, or is expressed at a lower level, in a tissue of interest in a young healthy individual. Conversely, a senescence-associated gene, can be one that is not expressed, or is expressed at a lower level, in a tissue of interest undergoing senescence than it is expressed in tissues of a healthy young subject.

“Amplification primers” are oligonucleotides comprising either natural or analog nucleotides that can serve as the basis for the amplification of a selected nucleic acid sequence. They include, for example, both polymerase chain reaction primers and ligase chain reaction oligonucleotides.

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv).

The term “biological samples” refers to any tissue or liquid sample having genomic DNA or other nucleic acids (e.g., mRNA) or proteins. It refers to samples of cells or tissue from a healthy young individual as well as samples of cells or tissue undergoing senescence or from an old individual.

The term “gene” means the segment of DNA involved in producing a polypeptide chain; it includes regions preceding and following the coding region (leader and trailer) as well as intervening sequences (introns) between individual coding segments (exons).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al., (1992); Rossolini et al., Mol. Cell. Probes 8:91-98 (1994)). The term nucleic acid is used interchangeably with gene, cDNA, and mRNA encoded by a gene.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well-known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 30% identity, optionally 40%, 50%, 60%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 15 amino acids in length, or more preferably over a region that is at least 30 amino acids in length.

The term “similarity,” or “percent similarity,” in the context of two or more polypeptide sequences, refer to two or more sequences or subsequences that have a specified percentage of amino acid residues that are either the same or similar as defined in the 8 conservative amino acid substitutions defined above (i.e., 30% identity, optionally 40%, 50%, 60%, 80%, 85%, 90%, or 95% similar over a specified region), when compared and aligned for maximum correspondence over a comparison window, or designated region as measured using one of the following sequence comparison algorithms or by manual alignment and visual inspection. Such sequences are then said to be “substantially similar.” Optionally, this identity exists over a region that is at least about 15 amino acids in length, or more preferably over a region that is at least about 30-60 amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman, Adv. Appl. Math. 2:482c (1970), by the homology alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Preferred examples of algorithm that is suitable for determining percent sequence identity and sequence similarity are the BLAST and BLAST 2.0 algorithms, which are described in Altschul et al., Nuc. Acids Res. 25:3389-3402 (1977), and Altschul et al., J. Mol. Biol. 215:403-410 (1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5× SSC, and 1% SDS, incubating at 42° C., or 5× SSC, 1% SDS, incubating at 65° C., with wash in 0.2× SSC, and 0.1% SDS at 65° C. Such washes can be performed for 5, 15, 30, 60, 120, or more minutes.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1× SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

As used herein a “nucleic acid probe” is defined as a nucleic acid capable of binding to a target nucleic acid (e.g., a nucleic acid encoding an aging-associated protein) of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions.

Nucleic acid probes can be DNA or RNA fragments. DNA fragments can be prepared, for example, by digesting plasmid DNA, or by use of PCR, or synthesized by either the phosphoramidite method described by Beaucage and Carruthers (Tetrahedron Lett. 22:1859-1862 (1981)), or by the triester method according to Matteucci et al. (J. Am. Chem. Soc. 103:3185 (1981)). A double-stranded fragment may then be obtained, if desired, by annealing the chemically synthesized single strands together under appropriate conditions, or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

A “labeled nucleic acid probe” is a nucleic acid probe that is bound, either covalently, through a linker, or through ionic, van der Waals or hydrogen bonds to a label such that the presence of the probe may be determined by detecting the presence of the label bound to the probe.

The phrase “a nucleic acid sequence encoding” refers to a nucleic acid which contains sequence information for a structural RNA such as rRNA, a tRNA, or the primary amino acid sequence of a specific protein or peptide, or a binding site for a transacting regulatory agent. This phrase specifically encompasses degenerate codons (i.e., different codons which encode a single amino acid) of the native sequence or sequences which may be introduced to conform with codon preference in a specific host cell.

The term “recombinant” when used with reference, e.g., to a cell, or to a nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under-expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “specifically (or selectively) binds to an antibody” or “specifically (or selectively) immunoreactive with,” when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised against a protein of the invention can be selected to obtain antibodies specifically immunoreactive with that protein and not with other proteins, except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane, Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise and more typically more than 10 to 100 times background.

“Non-proliferating cells” are those which are said to be in a Gₒ-phase where the cells are in a resting stage of arrested growth at the Gₒ phase, usually because they are deprived of an essential nutrient and cannot grow exponentially.

“Proliferating cells” are those which are actively undergoing cell division and grow exponentially.

### III. DETECTION OF GENE EXPRESSION AND GENOMIC ANALYSIS OF AGING-ASSOCIATED PROTEINS

The polynucleotides and polypeptides of the present invention can be employed as research reagents and materials for discovery of treatments and diagnostics to human disease. It will be readily apparent to those of skill in the art that although the following discussion is directed to methods for detecting nucleic acids associated with senescence, similar methods can be used to detect nucleic acids associated with cell proliferation, arrested cell growth, cell youthfulness and/or nucleic acids associated with aging-related diseases.

As should be apparent to those of skill in the art, the invention is the identification of aging-associated genes and the discovery that multiple nucleic acids are associated with aging. Accordingly, the present invention also includes methods for detecting the presence, alteration or absence of aging-associated nucleic acids (e.g., DNA or RNA) in a physiological specimen in order to determine the age of cells in vitro, or ex vivo and their level of activity, i.e., proliferation state or not, the genotype and risk of senescence or aging associated with mutations created in non-senescent sequences. Although any tissue having cells bearing the genome of an individual, or RNA associated with senescence, can be used, the most convenient specimen will be blood samples or biopsies of suspect tissue. It is also possible and preferred in some circumstances to conduct assays on cells that are isolated under microscopic visualization. A particularly useful method is the microdissection technique described in WO 95/23960. The cells isolated by microscopic visualization can be used in any of the assays described herein including both genomic and immunological based assays.

This invention provides for methods of genotyping family members in which relatives are diagnosed with premature aging, general aging and skin aging. Conventional methods of genotyping are provided herein.

The invention provides methods for detecting whether a cell is in a senescent state and/or is undergoing senescence. The methods typically comprise contacting RNA from the cell with a probe which comprises a polynucleotide sequence associated with aging, and determining whether the amount of the probe which hybridizes to the RNA is increased or decreased relative to the amount of the probe which hybridizes to RNA from a non-senescent cell. The assays are useful for detecting cell degeneration associated with, for example, aging-related diseases, such as Werner Syndrome and Progeria. One can also detect cell youthfulness or whether a cell is arrested at the G₀ stage of the cell cycle using the methods of the invention.

The probes are capable of binding to a target nucleic acid (e.g., a nucleic acid associated with senescence). By assaying for the presence or absence of the probe, one can detect the presence or absence of the target nucleic acid in a sample. Preferably, non-hybridizing probe and target nucleic acids are removed (e.g., by washing) prior to detecting the presence of the probe.

A variety of methods of specific DNA and RNA measurement using nucleic acid hybridization techniques are known to those of skill in the art (see, Sambrook, supra). For example, one method for evaluating the presence or absence of the DNA in a sample involves a Southern transfer. Briefly, the digested genomic DNA is run on agarose slab gels in buffer and transferred to membranes. Hybridization is carried out using the probes discussed above. Visualization of the hybridized portions allows the qualitative determination of the presence, alteration or absence of a senescence-associated gene.

Similarly, a Northern transfer may be used for the detection of aging-associated mRNA in samples of RNA from cells expressing the aging-associated proteins. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify the presence or absence of the subject protein transcript. Alternatively, the amount of, for example, a senescence-associated mRNA can be analyzed in the absence of electrophoretic separation.

The selection of a nucleic acid hybridization format is not critical. A variety of nucleic acid hybridization formats are known to those skilled in the art. For example, common formats include sandwich assays and competition or displacement assays. Hybridization techniques are generally described in Hames and Higgins, Nucleic Acid Hybridization, A Practical Approach, IRL Press (1985); Gall and Pardue, Proc. Natl. Acad. Sci. U.S.A. 63:378-383 (1969); and John et al., Nature, 223:582-587 (1969).

For example, sandwich assays are commercially useful hybridization assays for detecting or isolating nucleic acids. Such assays utilize a “capture” nucleic acid covalently immobilized to a solid support and a labeled “signal” nucleic acid in solution. The clinical sample will provide the target nucleic acid. The “capture” nucleic acid and “signal” nucleic acid probe hybridize with the target nucleic acid to form a “sandwich” hybridization complex. To be effective, the signal nucleic acid cannot hybridize with the capture nucleic acid.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label (see, e.g., Tijssen, “Practice and Theory of Enzyme Immunoassays,” Laboratory Techniques in Biochemistry and Molecular Biology, Burdon and van Knippenberg Eds., Elsevier (1985), pp. 9-20).

The probes are typically labeled either directly, as with isotopes, chromophores, lumiphores, chromogens, or indirectly, such as with biotin, to which a streptavidin complex may later bind. The detectable labels used in the assays of the present invention can be primary labels (where the label comprises an element that is detected directly or that produces a directly detectable element) or secondary labels (where the detected label binds to a primary label, e.g., as is common in immunological labeling). Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labeled probes or the like.

Other labels include, e.g., ligands which bind to labeled antibodies, fluorophores, chemi-luminescent agents, enzymes, and antibodies which can serve as specific binding pair members for a labeled ligand. An introduction to labels, labeling procedures and detection of labels is found in Polak and Van Noorden, Introduction to Immunocytochemistry, 2nd ed., Springer Verlag, NY (1997); and in Haugland, Handbook of Fluorescent Probes and Research Chemicals, a combined handbook and catalogue Published by Molecular Probes, Inc. (1996). Primary and secondary labels can include undetected elements as well as detected elements. Useful primary and secondary labels in the present invention can include spectral labels such as fluorescent dyes (e.g., fluorescein and derivatives such as fluorescein isothiocyanate (FITC) and Oregon Green™, rhodamine and derivatives (e.g., Texas red, tetrarhodimine isothiocynate (TRITC), etc.), digoxigenin, biotin, phycoerythrin, AMCA, CyDyes™, and the like), radiolabels (e.g., ³H, ¹²⁵I, ³⁵I, ¹⁴C, ³²P, ³³P, etc.), enzymes (e.g., horse radish peroxidase, alkaline phosphatase, etc.), spectral colorimetric labels such as colloidal gold or colored glass or plastic (e.g., polystyrene, polypropylene, latex, etc.) beads. The label may be coupled directly or indirectly to a component of the detection assay (e.g., the probe) according to methods well known in the art. As indicated above, a wide variety of labels may be used, with the choice of label depending on the sensitivity required, the ease of conjugation with the compound, stability requirements, available instrumentation, and disposal provisions.

Preferred labels include those that use: 1) chemiluminescence (using horseradish peroxidase and/or alkaline phosphatase with substrates that produce photons as breakdown products as described above) with kits being available, e.g., from Molecular Probes, Amersham, Boehringer-Mannheim, and Life Technologies/Gibco BRL; 2) color production (using both horseradish peroxidase and/or alkaline phosphatase with substrates that produce a colored precipitate [kits available from Life Technologies/Gibco BRL, and Boehringer-Mannheim]); 3) hemifluorescence using, e.g., alkaline phosphatase and the substrate AttoPhos [Amersham] or other substrates that produce fluorescent products; 4) fluorescence (e.g., using Cy-5 [Amersham]), fluorescein, and other fluorescent tags); and 5) radioactivity. Other methods for labeling and detection will be readily apparent to one skilled in the art.

Preferred enzymes that can be conjugated to detection reagents of the invention include, e.g., β-galactosidase, luciferase, horse radish peroxidase, and alkaline phosphatase. The chemiluminescent substrate for luciferase is luciferin. One embodiment of a chemiluminescent substrate for β-galactosidase is 4-methylumbelliferyl-β-D-galactoside. Embodiments of alkaline phosphatase substrates include p-nitrophenyl phosphate (pNPP), which is detected with a spectrophotometer; 5-bromo-4-chloro-3-indolyl phosphate/nitro blue tetrazolium (BCIP/NBT) and fast red/napthol AS-TR phosphate, which are detected visually; and 4-methoxy-4-(3-phosphonophenyl) spiro[1,2-dioxetane-3,2′-adamantane], which is detected with a luminometer. Embodiments of horse radish peroxidase substrates include 2,2′azino-bis(3-ethylbenzthiazoline-6 sulfonic acid) (ABTS), 5-aminosalicylic acid (5AS), o-dianisidine, and o-phenylenediamine (OPD), which are detected with a spectrophotometer; and 3,3,5,5′-tetramethylbenzidine (TMB), 3,3′diaminobenzidine (DAB), 3-amino-9-ethylcarbazole (AEC), and 4-chloro-1-naphthol (4C1N), which are detected visually. Other suitable substrates are known to those skilled in the art. The enzyme-substrate reaction and product detection are performed according to standard procedures well known to those skilled in the art and kits for performing enzyme immunoassays are available as described herein.

In general, a detector which monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill in the art. Commonly, an optical image of a substrate comprising bound labeling moieties is digitized for subsequent computer analysis.

Most typically, the amount of, for example, an aging-associated RNA is measured by quantitating the amount of label fixed to the solid support by binding of the detection reagent. Typically, the presence of a modulator during incubation will increase or decrease the amount of label fixed to the solid support relative to a control incubation which does not comprise the modulator, or as compared to a baseline established for a particular reaction type. Means of detecting and quantitating labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes and many other detection systems which are widely available.

In preferred embodiments, the target nucleic acid or the probe is immobilized on a solid support. Solid supports suitable for use in the assays of the invention are known to those of skill in the art. As used herein, a solid support is a matrix of material in a substantially fixed arrangement. Exemplar solid supports include glasses, plastics, polymers, metals, metalloids, ceramics, organics, etc. Solid supports can be flat or planar, or can have substantially different conformations. For example, the substrate can exist as particles, beads, strands, precipitates, gels, sheets, tubing, spheres, containers, capillaries, pads, slices, films, plates, dipsticks, slides, etc. Magnetic beads or particles, such as magnetic latex beads and iron oxide particles, are examples of solid substrates that can be used in the methods of the invention. Magnetic particles are described in, for example, U.S. Pat. No. 4,672,040, and are commercially available from, for example, PerSeptive Biosystems, Inc. (Framingham Mass.), Ciba Coming (Medfield Mass.), Bangs Laboratories (Carmel Ind.), and BioQuest, Inc. (Atkinson N.H.). The substrate is chosen to maximize signal to noise ratios, primarily to minimize background binding, for ease of washing and cost.

A variety of automated solid-phase assay techniques are also appropriate. For instance, very large scale immobilized polymer arrays (VLSIPS™), available from Affymetrix, Inc. (Santa Clara, Calif.) can be used to detect changes in expression levels of a plurality of aging-associated nucleic acids simultaneously (see, Tijssen, supra.; Fodor et al., Science 251:767-777 (1991); Sheldon et al., Clinical Chemistry 39(4):718-719 (1993); and Kozal et al., Nature Medicine 2(7):753-759 (1996)). Thus, in one embodiment, the invention provides methods for detecting the expression levels of senescence-associated nucleic acids in which nucleic acids (e.g., RNA from a cell culture) are hybridized to an array of nucleic acids that are known to be associated with aging. For example, in the assay described supra, oligonucleotides which hybridize to a plurality of senescence-associated nucleic acids are optionally synthesized on a DNA chip (such chips are available from Affymetrix) and the RNA from a biological sample, such as a cell culture, is hybridized to the chip for simultaneous analysis of multiple senescence-associated nucleic acids. The aging-associated nucleic acids that are present in the sample which is assayed are detected at specific positions on the chip.

Detection can be accomplished, for example, by using a labeled detection moiety that binds specifically to duplex nucleic acids (e.g., an antibody that is specific for RNA-DNA duplexes). One preferred example uses an antibody that recognizes DNA-RNA heteroduplexes in which the antibody is linked to an enzyme (typically by recombinant or covalent chemical bonding). The antibody is detected when the enzyme reacts with its substrate, producing a detectable product. Coutlee et al., Analytical Biochemistry 181:153-162 (1989); Bogulavski et al., J. Immunol. Methods 89:123-130 (1986); Prooijen-Knegt, Exp. Cell Res. 141:397-407 (1982); Rudkin, Nature 265:472-473 (1976); Stollar, PNAS 65:993-1000 (1970); Ballard, Mol. Immunol. 19:793-799 (1982); Pisetsky and Caster, Mol. Immunol. 19:645-650 (1982); Viscidi et al., J. Clin. Microbial. 41:199-209 (1988); and Kiney et al., J. Clin. Microbiol. 27:6-12 (1989) describe antibodies to RNA duplexes, including homo and heteroduplexes. Kits comprising antibodies specific for DNA:RNA hybrids are available, e.g., from Digene Diagnostics, Inc. (Beltsville, Md.).

In addition to available antibodies, one of skill in the art can easily make antibodies specific for nucleic acid duplexes using existing techniques, or modify those antibodies which are commercially or publicly available. In addition to the art referenced above, general methods for producing polyclonal and monoclonal antibodies are known to those of skill in the art (see, e.g., Paul (ed), Fundamental Immunology, Third Edition Raven Press, Ltd., NY (1993); Coligan, Current Protocols in Immunology Wiley/Greene, NY (1991); Harlow and Lane, Antibodies: A Laboratory Manual Cold Spring Harbor Press, NY (1989); Stites et al., Basic and Clinical Immunology (4th ed.) Lange Medical Publications, Los Altos, Calif., and references cited therein; Goding, Monoclonal Antibodies: Principles and Practice (2d ed.) Academic Press, New York, N.Y., (1986); and Kohler and Milstein, Nature 256: 495-497 (1975)). Other suitable techniques for antibody preparation include selection of libraries of recombinant antibodies in phage or similar vectors (see, Huse et al., Science 246:1275-1281 (1989); and Ward et al., Nature 341:544-546 (1989)). Specific monoclonal and polyclonal antibodies and antisera will usually bind with a K_{D} of at least about 0.1 μM, preferably at least about 0.01 μM or better, and most typically and preferably, 0.001 μM or better.

The nucleic acids used in this invention can be either positive or negative probes. Positive probes bind to their targets and the presence of duplex formation is evidence of the presence of the target. Negative probes fail to bind to the suspect target and the absence of duplex formation is evidence of the presence of the target. For example, the use of a wild type specific nucleic acid probe or PCR primers may serve as a negative probe in an assay sample where only the nucleotide sequence of interest is present.

The sensitivity of the hybridization assays may be enhanced through the use of a nucleic acid amplification system which multiplies the target nucleic acid being detected. Examples of such systems include the polymerase chain reaction (PCR) system and the ligase chain reaction (LCR) system. Other methods recently described in the art are the nucleic acid sequence based amplification (NASBAθ, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation.

A preferred embodiment is the use of allelic specific amplifications. In the case of PCR, the amplification primers are designed to bind to a portion of, for example, a gene encoding an senescence-associated protein, but the terminal base at the 3′ end is used to discriminate between the mutant and wild-type forms of the senescence-associated protein gene. If the terminal base matches the point mutation or the wild-type, polymerase dependent three prime extension can proceed and an amplification product is detected. This method for detecting point mutations or polymorphisms is described in detail by Sommer et al., in Mayo Clin. Proc. 64:1361-1372 (1989). By using appropriate controls, one can develop a kit having both positive and negative amplification products. The products can be detected using specific probes or by simply detecting their presence or absence. A variation of the PCR method uses LCR where the point of discrimination, i.e., either the point mutation or the wild-type bases fall between the LCR oligonucleotides. The ligation of the oligonucleotides becomes the means for discriminating between the mutant and wild-type forms of the gene encoding senescence-associated protein.

An alternative means for determining the level of expression of the nucleic acids of the present invention is in situ hybridization. In situ hybridization assays are well known and are generally described in Angerer et al., Methods Enzymol. 152:649-660 (1987). In an in situ hybridization assay, cells, preferentially human cells, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

### IV. IMMUNOLOGICAL DETECTION OF AN AGING-ASSOCIATED PROTEIN

In addition to the detection of the subject protein gene expression using nucleic acid hybridization technology, one can also use immunoassays to detect the protein itself. Immunoassays can be used to qualitatively or quantitatively analyze the proteins of interest. A general overview of the applicable technology can be found in Harlow and Lane, Antibodies: A Laboratory Manual, Cold Spring Harbor Pubs., NY (1988). Although the following discussion is directed to methods for detecting target proteins associated with aging, similar methods can be used to detect target proteins associated with, e.g., cell proliferation, arrested cell growth, cell youthfulness and/or nucleic acids associated with aging-related diseases (e.g., Werner Syndrome, Progeria, neoplasms, etc.).

### A. Antibodies to Target Proteins

Methods for producing polyclonal and monoclonal antibodies that react specifically with a protein of interest are known to those of skill in the art (see, e.g., Coligan, supra; and Harlow and Lane, supra; Stites et al., supra and references cited therein; Goding, supra; and Kohler and Milstein, Nature 256:495-497 (1975)). Such techniques include antibody preparation by selection of antibodies from libraries of recombinant antibodies in phage or similar vectors (see, Huse et al., supra; and Ward et al., supra). For example, in order to produce antisera for use in an immunoassay, the protein of interest or an antigenic fragment thereof, is isolated as described herein. For example, a recombinant protein is produced in a transformed cell line. An inbred strain of mice or rabbits is immunized with the protein using a standard adjuvant, such as Freund's adjuvant, and a standard immunization protocol. Alternatively, a synthetic peptide derived from the sequences disclosed herein and conjugated to a carrier protein can be used as an immunogen.

Polyclonal sera are collected and titered against the immunogen protein in an immunoassay, for example, a solid phase immunoassay with the immunogen immobilized on a solid support. Polyclonal antisera with a titer of 10⁴ or greater are selected and tested for their cross-reactivity against non-aging-associated proteins or even other homologous proteins from other organisms, using a competitive binding immunoassay. Specific monoclonal and polyclonal antibodies and antisera will usually bind with a K_{D} of at least about 0.1 mM, more usually at least about 1 82 M, preferably at least about 0.1 μM or better, and most preferably, 0.01 μM or better.

A number of proteins of the invention comprising immunogens may be used to produce antibodies specifically or selectively reactive with the proteins of interest. Recombinant protein is the preferred immunogen for the production of monoclonal or polyclonal antibodies. Naturally occurring protein may also be used either in pure or impure form. Synthetic peptides made using the protein sequences described herein may also be used as an immunogen for the production of antibodies to the protein. Recombinant protein can be expressed in eukaryotic or prokaryotic cells and purified as generally described infra. The product is then injected into an animal capable of producing antibodies. Either monoclonal or polyclonal antibodies may be generated for subsequent use in immunoassays to measure the protein.

Methods of production of polyclonal antibodies are known to those of skill in the art. In brief, an immunogen, preferably a purified protein, is mixed with an adjuvant and animals are immunized. The animal's immune response to the immunogen preparation is monitored by taking test bleeds and determining the titer of reactivity to the senescence protein of interest. When appropriately high titers of antibody to the immunogen are obtained, blood is collected from the animal and antisera are prepared. Further fractionation of the antisera to enrich for antibodies reactive to the protein can be done if desired (see, Harlow and Lane, supra).

Monoclonal antibodies may be obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519 (1976)). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., supra.

Once target protein specific antibodies are available, the protein can be measured by a variety of immunoassay methods with qualitative and quantitative results available to the clinician. For a review of immunological and immunoassay procedures in general see, Stites, supra. Moreover, the immunoassays of the present invention can be performed in any of several configurations, which are reviewed extensively in Maggio, Enzyme Immunoassay, CRC Press, Boca Raton, Fla. (1980); Tijssen, supra; and Harlow and Lane, supra.

Immunoassays to measure target proteins in a human sample may use a polyclonal antiserum raised to the protein partially encoded by a sequence described herein or a fragment thereof. This antiserum is selected to have low cross-reactivity against non-senescence-associated proteins and any such cross-reactivity is removed by immunoabsorption prior to use in the immunoassay.

In order to produce antisera for use in an immunoassay, the aging-associated protein of interest or a fragment thereof, for example, is isolated as described herein. For example, recombinant protein is produced in a transformed cell line. An inbred strain of mice, such as Balb/c, is immunized with the protein or a peptide using a standard adjuvant, such as Freund's adjuvant, and a standard mouse immunization protocol. Alternatively, a synthetic peptide derived from the sequences disclosed herein and conjugated to a carrier protein can be used as an immunogen. Polyclonal sera are collected and titered against the immunogen protein in an immunoassay, such as, for example, a solid phase immunoassay with the immunogen immobilized on a solid support. Polyclonal antisera with a titer of 10⁴ or greater are selected and tested for their cross-reactivity against non-aging-associated proteins, using a competitive binding immunoassay such as the one described in Harlow and Lane, supra, at pages 570-573 and below.

### B. Immunological Binding Assays

In a preferred embodiment, a protein of interest is detected and/or quantified using any of a number of well known immunological binding assays (see, e.g., U.S. Pat. Nos. 4,366,241; 4,376,110; 4,517,288; and 4,837,168). For a review of the general immunoassays, see also Asai, Methods in Cell Biology Volume 37: Antibodies in Cell Biology, Academic Press, Inc. NY (1993); and Stites and Terr, supra. Immunological binding assays (or immunoassays) typically utilize a “capture agent” to specifically bind to and often immobilize the analyte (e.g., the aging-associated protein or antigenic subsequence thereof). The capture agent is a moiety that specifically binds to the analyte. In a preferred embodiment, the capture agent is an antibody that specifically binds, for example, the aging-associated protein. The antibody (e.g., anti-aging-associated protein antibody) may be produced by any of a number of means well known to those of skill in the art and as described above.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled aging-associated protein polypeptide or a labeled anti-aging-associated protein antibody. Alternatively, the labeling agent may be a third moiety, such as another antibody, that specifically binds to the antibody/protein complex.

In a preferred embodiment, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval et al., J. Immunol. 111: 1401-1406 (1973); and Akerstrom et al., J. Immunol. 135:2589-2542 (1985)).

Throughout the assays, incubation and/or washing steps may be required after each combination of reagents. Incubation steps can vary from about 5 seconds to several hours, preferably from about 5 minutes to about 24 hours. The incubation time will depend upon the assay format, analyte, volume of solution, concentrations, and the like. Usually, the assays will be carried out at ambient temperature, although they can be conducted over a range of temperatures, such as 10° C. to 40° C.

1. Non-competitive Assay Formats

Immunoassays for detecting proteins of interest from tissue samples may be either competitive or noncompetitive. Noncompetitive immunoassays are assays in which the amount of captured analyte (in this case the protein) is directly measured. In one preferred “sandwich” assay, for example, the capture agent (e.g., anti-aging-associated protein antibodies) can be bound directly to a solid substrate where it is immobilized. These immobilized antibodies then capture the aging-associated protein present in the test sample. The aging-associated protein thus immobilized is then bound by a labeling agent, such as a second anti-aging-associated protein antibody bearing a label. Alternatively, the second antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin.

2. Competitive Assay Formats

In competitive assays, the amount of target protein (analyte) present in the sample is measured indirectly by measuring the amount of an added (exogenous) analyte (e.g., the aging-associated protein of interest) displaced (or competed away) from a capture agent (anti-aging-associated protein antibody) by the analyte present in the sample. In one competitive assay, a known amount of, in this case, the protein of interest is added to the sample and the sample is then contacted with a capture agent, in this case an antibody that specifically binds to the aging-associated protein. The amount of aging-associated protein bound to the antibody is inversely proportional to the concentration of aging-associated protein present in the sample. In a particularly preferred embodiment, the antibody is immobilized on a solid substrate. The amount of the aging-associated protein bound to the antibody may be determined either by measuring the amount of subject protein present in an aging-associated protein/antibody complex or, alternatively, by measuring the amount of remaining uncomplexed protein. The amount of aging-associated protein may be detected by providing a labeled aging-associated protein molecule.

A hapten inhibition assay is another preferred competitive assay. In this assay, a known analyte, in this case the target protein, is immobilized on a solid substrate. A known amount of anti-aging-associated protein antibody is added to the sample, and the sample is then contacted with the immobilized target. In this case, the amount of anti-aging-associated protein antibody bound to the immobilized aging-associated protein is inversely proportional to the amount of aging-associated protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Immunoassays in the competitive binding format can be used for cross-reactivity determinations. For example, the protein encoded by the sequences described herein can be immobilized on a solid support. Proteins are added to the assay which compete with the binding of the antisera to the immobilized antigen. The ability of the above proteins to compete with the binding of the antisera to the immobilized protein is compared to that of the protein encoded by any of the sequences described herein. The percent cross-reactivity for the above proteins is calculated, using standard calculations. Those antisera with less than 10% cross-reactivity with each of the proteins listed above are selected and pooled. The cross-reacting antibodies are optionally removed from the pooled antisera by immunoabsorption with the considered proteins, e.g., distantly related homologues.

The immunoabsorbed and pooled antisera are then used in a competitive binding immunoassay as described above to compare a second protein, thought to be perhaps the protein of the present invention, to the immunogen protein. In order to make this comparison, the two proteins are each assayed at a wide range of concentrations and the amount of each protein required to inhibit 50% of the binding of the antisera to the immobilized protein is determined. If the amount of the second protein required is less than 10 times the amount of the protein partially encoded by a sequence herein that is required, then the second protein is said to specifically bind to an antibody generated to an immunogen consisting of the target protein.

3. Other Assay Formats

In a particularly preferred embodiment, Western blot (immunoblot) analysis is used to detect and quantify the presence of aging-associated protein in the sample. The technique generally comprises separating sample proteins by gel electrophoresis on the basis of molecular weight, transferring the separated proteins to a suitable solid support (such as, e.g., a nitrocellulose filter, a nylon filter, or a derivatized nylon filter) and incubating the sample with the antibodies that specifically bind the protein of interest. For example, anti-aging-associated protein antibodies specifically bind to the aging-associated protein on the solid support. These antibodies may be directly labeled or alternatively may be subsequently detected using labeled antibodies (e.g., labeled sheep anti-mouse antibodies) that specifically bind to the antibodies against the protein of interest.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev. 5:34-41 (1986)).

4. Reduction of Non-Specific Binding

One of skill in the art will appreciate that it is often desirable to use non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this involves coating the substrate with a proteinaceous composition. In particular, protein compositions, such as bovine serum albumin (BSA), nonfat powdered milk and gelatin, are widely used with powdered milk being most preferred.

5. Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most labels useful in such methods can be applied to the present invention. Thus, a label is any composition detectable by spectroscopic, photochemical, biochemical, immunochemical, electrical, optical or chemical means. Useful labels in the present invention include magnetic beads (e.g., Dynabead™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads.

The label may be coupled directly or indirectly to the desired component of the assay according to methods well known in the art. As indicated above, a wide variety of labels may be used, with the choice of label depending on the sensitivity required, the ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Thyroxine, and cortisol can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol (for a review of various labeling or signal producing systems which may be used, see, U.S. Pat. No. 4,391,904).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected directly by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need to be labeled and the presence of the target antibody is detected by simple visual inspection.

### V. SCREENING FOR MODULATORS OF THE AGING PROCESS

The invention also provides methods for identifying compounds that modulate the aging process, e.g., the senescence of a cell. For example, the methods can identify compounds that increase or decrease the expression level of genes associated with aging (e.g., senescence, cell proliferation, arrested cell growth, cell youthfulness, etc.) and aging-related conditions. Although the following discussion is directed to methods for screening for modulators of senescence, similar methods can be used to screen for modulators of, e.g., cell proliferation, cell growth, cell youthfulness and/or expression of nucleic acids associated with aging-related diseases (e.g., Werner Syndrome, Progeria, etc.).

For instance, compounds that are identified as modulators of senescence using the methods of the invention find use both in vitro and in vivo. For example, one can treat cell cultures with the modulators in experiments designed to determine the mechanisms by which senescence is regulated. Compounds that decrease or delay senescence are useful for extending the useful life of cell cultures that are used for production of biological products such as recombinant proteins. In vivo uses of compounds that delay cell senescence include, for example, delaying the aging process and treating conditions associated with premature aging. Conversely, compounds that accelerate or increase cell senescence are useful as anticancer agents, as cancer is often associated with a loss of a cell's ability to undergo normal senescence.

The methods typically involve culturing a cell in the presence of a potential modulator to form a first cell culture. RNA (or cDNA) from the first cell culture is contacted with a probe which comprises a polynucleotide sequence associated with aging. The amount of the probe which hybridizes to the RNA (or cDNA) from the first cell culture is determined. Typically, one determines whether the amount of probe which hybridizes to the RNA (or cDNA) is increased or decreased relative to the amount of the probe which hybridizes to RNA (or cDNA) from a second cell culture grown in the absence of the modulator.

It may be further determined whether the modulator-induced increase or decrease in RNA (or cDNA) levels of the target sequence is correlated with any age-associated change in cellular phenotype. For example, a cell population that is treated with a modulator which induces decreased expression of a gene that is normally upregulated with aging or a cell that is treated with a modulator which induces increased expression of a gene that is normally downregulated with aging may be further tested for, e.g., regained proliferative potential, which is reflective of a “younger” phenotype. Frequently, a young phenotype is the phenotype observed in cells or tissues that are obtained from an individual of about 30 years or less in age, whereas an aged phenotype is the phenotype observed in cells or tissues that are obtained from an individual of about 65 years or more in age.

Essentially any chemical compound can be used as a potential modulator in the assays of the invention, although most often compounds that can be dissolved in aqueous or organic (for example, DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial library containing a large number of potential therapeutic compounds (potential modulator compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res. 37:487-493 (1991); and Houghton et al., Nature 354:84-88 (1991)). Other chemistries for generating chemical diversity libraries can also be used. Such chemistries include, but are not limited to: peptoids (WO 91/19735), encoded peptides (WO 93/20242), random bio-oligomers (WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel et al., Current Protocols in Molecular Biology (1987); Berger et al., supra; and Sambrook et al., supra), peptide nucleic acid libraries (see, e.g., U.S. Pat. No. 5,539,083), antibody libraries (see, e.g., Vaughn et al., Nature Biotechnology, 14(3):309-314 (1996); and PCT/US96/10287), carbohydrate libraries (see, e.g., Liang et al., Science, 274:1520-1522 (1996); and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

As noted, the invention provides in vitro assays for identifying, in a high throughput format, compounds that can modulate the cell senescence. Control reactions that measure the senescence level of the cell in a reaction that does not include a potential modulator are optional, as the assays are highly uniform. Such optional control reactions are appropriate and increase the reliability of the assay. Accordingly, in a preferred embodiment, the methods of the invention include such a control reaction. For each of the assay formats described, “no modulator” control reactions which do not include a modulator provide a background level of binding activity.

In some assays it will be desirable to have positive controls to ensure that the components of the assays are working properly. At least two types of positive controls are appropriate. First, a known activator of senescence can be incubated with one sample of the assay, and the resulting increase in signal resulting from an increased expression level of a gene associated with senescence determined according to the methods herein. Second, a known inhibitor of senescence can be added, and the resulting decrease in signal for the expression of a gene associated with senescence similarly detected. It will be appreciated that modulators can also be combined with activators or inhibitors to find modulators which inhibit the increase or decrease that is otherwise caused by the presence of the known modulator of the aging process.

In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (96) modulators. If 1536 well plates are used, then a single plate can easily assay from about 100 to about 1500 different compounds. It is possible to assay many different plates per day; assay screens for up to about 6,000-20,000, and even up to about 100,000 different compounds are possible using the integrated systems of the invention.

### VI. COMPOSITIONS, KITS AND INTEGRATED SYSTEMS

The invention provides compositions, kits and integrated systems for practicing the assays described herein. Although the following discussion is directed to kits for carrying out assays using nucleic acids (or proteins, antibodies, etc.) associated with aging, similar kits can be assembled for carrying out assays using nucleic acids (or proteins, antibodies, etc.) associated with cell proliferation, cell youthfulness, arrested cell growth and/or nucleic acids associated with aging-related diseases (e.g., Werner Syndrome, Progeria, neoplasms, etc.). For instance, an assay composition having a nucleic acid associated with aging and a labeling reagent is provided by the present invention. In preferred embodiments, a plurality of, for example, aging-associated nucleic acids are provided in the assay compositions. The invention also provides assay compositions for use in solid phase assays; such compositions can include, for example, one or more aging-associated nucleic acids immobilized on a solid support, and a labeling reagent. In each case, the assay compositions can also include additional reagents that are desirable for hybridization. Modulators of expression of, for example, aging-associated nucleic acids can also be included in the assay compositions.

The invention also provides kits for carrying out the assays of the invention. The kits typically include a probe which comprises a polynucleotide sequence associated with senescence, and a label for detecting the presence of the probe. Preferably, the kits will include a plurality of polynucleotide sequences associated with aging. Kits can include any of the compositions noted above, and optionally further include additional components such as instructions to practice a high-throughput method of assaying for an effect on cell proliferation and transformation and expression of aging-associated genes, one or more containers or compartments (e.g., to hold the probe, labels, or the like), a control modulator of the aging process, a robotic armature for mixing kit components or the like.

The invention also provides integrated systems for high-throughput screening of potential modulators for an effect on the aging process. The systems typically include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety.

A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous STAT binding assays.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image, e.g., using PC (Intel x86 or Pentium chip-compatible DOS®, OS2® WINDOWS®, WINDOWS NT® or WINDOWS95® based computers), MACINTOSH®, or UNIX® based (e.g., SUN® work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

### VII. GENE THERAPY APPLICATIONS

A variety of human diseases can be treated by therapeutic approaches that involve stably introducing a gene into a human cell such that the gene is transcribed and the gene product is produced in the cell. Diseases amenable to treatment by this approach include inherited diseases, including those in which the defect is in a single gene. Gene therapy is also useful for treatment of acquired diseases and other conditions. For discussions on the application of gene therapy towards the treatment of genetic as well as acquired diseases, see, Miller, Nature 357:455-460 (1992); and Mulligan, Science 260:926-932 (1993).

### A. Vectors for Gene Delivery

For delivery to a cell or organism, the nucleic acids of the invention can be incorporated into a vector. Examples of vectors used for such purposes include expression plasmids capable of directing the expression of the nucleic acids to the target cell. In other instances, the vector is a viral vector system wherein the nucleic acids are incorporated into a viral genome that is capable of transfecting the target cell. In a preferred embodiment, the nucleic acids can be operably linked to expression and control sequences that can direct the expression of the gene in the desired target host cells. Thus, one can achieve expression of the nucleic acid under appropriate conditions in the target cell.

### B. Gene Delivery Systems

Viral vector systems useful in the expression of the nucleic acids include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, retroviruses (including but not limited to Rous sarcoma virus), and MoMLV. Typically, the genes of interest are inserted into such vectors to allow packaging of the gene construct, typically with accompanying viral DNA, followed by infection of a sensitive host cell and expression of the gene of interest.

As used herein, “gene delivery system” refers to any means for the delivery of a nucleic acid of the invention to a target cell. In some embodiments of the invention, nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acids of the invention can be modified by the addition of receptor ligands or antibodies specific for a receptor to permit receptor-mediated endocytosis into specific cells (see, e.g., WO 93/20221; WO 93/14188; and WO 94/06923). In some embodiments of the invention, the DNA constructs of the invention are linked to viral proteins, such as adenovirus particles, to facilitate endocytosis (Curiel et al., Proc. Natl. Acad. Sci. U.S.A. 88:8850-8854 (1991)). In other embodiments, molecular conjugates of the instant invention can include microtubule inhibitors (WO 94/06922), synthetic peptides mimicking influenza virus hemagglutinin (Plank et al., J. Biol. Chem. 269:12918-12924 (1994)), and nuclear localization signals such as SV40 T antigen (WO 93/19768).

Retroviral vectors are also useful for introducing the nucleic acids of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site). See, Mulligan, In: Experimental Manipulation of Gene Expression, Inouye (ed), 155-173 (1983); Mann et al., Cell 33:153-159 (1983); and Cone and Mulligan, Proc. Nat. Acad. Sci. U.S.A. 81:6349-6353 (1984).

The design of retroviral vectors is well known to those of ordinary skill in the art. In brief, if the sequences necessary for encapsidation (or packaging of retroviral RNA into infectious virions) are missing from the viral genome, the result is a cis acting defect which prevents encapsidation of genomic RNA. The resulting mutant is, however, still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent application EPA 0 178 220; U.S. Pat. No. 4,405,712; Gilboa, Biotechniques 4:504-512 (1986); Mann et al., Cell 33:153-159 (1983); Cone and Mulligan, Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and the International publication No. WO 92/07943 entitled “Retroviral Vectors Useful in Gene Therapy.”

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell, but is capable of integrating into the host cell genome as a proviral sequence containing the desired nucleotide sequence. As a result, the patient is capable of producing, for example, the aging-associated protein and thus restore the cells to a normal, non-senescent, or, for example, non-cancerous phenotype.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see, e.g., Miller et al., J. Virol. 65:2220-2224 (1991)). Examples of other packaging cell lines are described in Cone and Mulligan, supra; Danos and Mulligan, Proc. Natl. Acad. Sci. USA 85:6460-6464 (1988); Eglitis et al. (1988), supra; and Miller (1990), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

In some embodiments of the invention, an antisense nucleic acid is administered which hybridizes to a gene associated with senescence or to a transcript thereof. The antisense nucleic acid can be provided as an antisense oligonucleotide (see, e.g., Murayama et al., Antisense Nucleic Acid Drug Dev. 7:109-114 (1997)). Genes encoding an antisense nucleic acid can also be provided; such genes can be introduced into cells by methods known to those of skill in the art. For example, one can introduce a gene that encodes an antisense nucleic acid in a viral vector, such as, for example, in hepatitis B virus (see, e.g., Ji et al., J. Viral Hepat. 4:167-173 (1997)), in adeno-associated virus (see, e.g., Xiao et al., Brain Res. 756:76-83 (1997)), or in other systems including, but not limited, to an HVJ (Sendai virus)-liposome gene delivery system (see, e.g., Kaneda et al., Ann. NY Acad. Sci. 811:299-308 (1997)), a “peptide vector” (see, e.g., Vidal et al., CR Acad. Sci III 32:279-287 (1997)), as a gene in an episomal or plasmid vector (see, e.g., Cooper et al., Proc. Natl. Acad. Sci. U.S.A. 94:6450-6455 (1997), Yew et al., Hum Gene Ther. 8:575-584 (1997)), as a gene in a peptide-DNA aggregate (see, e.g., Niidome et al., J. Biol. Chem. 272:15307-15312 (1997)), as “naked DNA” (see, e.g., U.S. Pat. Nos. 5,580,859 and 5,589,466), in lipidic vector systems (see, e.g., Lee et al., Crit Rev Ther Drug Carrier Syst. 14:173-206 (1997)), polymer coated liposomes (U.S. Pat. Nos. 5,213,804 and 5,013,556), cationic liposomes (U.S. Pat. Nos. 5,283,185; 5,578,475; 5,279,833; and 5,334,761), gas filled microspheres (U.S. Pat. No. 5,542,935), ligand-targeted encapsulated macromolecules (U.S. Pat. Nos. 5,108,921; 5,521,291; 5,554,386; and 5,166,320).

### C. Pharmaceutical Formulations

When used for pharmaceutical purposes, the vectors used for gene therapy are formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al., Biochemistry 5:467 (1966).

The compositions can additionally include a stabilizer, enhancer or other pharmaceutically acceptable carriers or vehicles. A pharmaceutically acceptable carrier can contain a physiologically acceptable compound that acts, for example, to stabilize the nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known in the art and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

### D. Administration of Formulations

The formulations of the present invention can be delivered to any tissue or organ using any delivery method known to the ordinarily skilled artisan. In some embodiments of the invention, the nucleic acids of the invention are formulated in mucosal, topical, and/or buccal formulations, particularly mucoadhesive gel and topical gel formulations. Exemplary permeation enhancing compositions, polymer matrices, and mucoadhesive gel preparations for transdermal delivery are disclosed in U.S. Pat. No. 5,346,701. In some embodiments of the invention, a therapeutic agent is formulated in ophthalmic formulations for administration to the eye.

### E. Methods of Treatment

The gene therapy formulations of the invention are typically administered to a cell. The cell can be provided as part of a tissue, such as an epithelial membrane, or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acids of the invention are introduced into cells by such methods as microinjection, calcium phosphate precipitation, liposome fusion, or biolistics. In further embodiments, the nucleic acids are taken up directly by the tissue of interest.

In some embodiments of the invention, the nucleic acids of the invention are administered ex vivo to cells or tissues explanted from a patient, then returned to the patient. Examples of ex vivo administration of therapeutic gene constructs include Arteaga et al., Cancer Research 56(5):1098-1103 (1996); Nolta et al., Proc Natl. Acad. Sci. USA 93(6):2414-9 (1996); Koc et al., Seminars in Oncology 23(1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

### VIII. GENERAL RECOMBINANT NUCLEIC ACIDS METHODS FOR USE WITH THE INVENTION

### A. General Recombinant Nucleic Acids Methods

Nucleotide sizes are given in either kilobases (kb) or base pairs (bp). These are estimates derived from agarose or acrylamide gel electrophoresis or, alternatively, from published DNA sequences.

Oligonucleotides that are not commercially available can be chemically synthesized according to the solid phase phosphoramidite triester method first described by Beaucage and Caruthers, Tetrahedron Letts., 22(20):1859-1862 (1981), using an automated synthesizer, as described in Needham Van Devanter et al., Nucleic Acids Res. 12:6159-6168 (1984). Purification of oligonucleotides is, for example, by either native acrylamide gel electrophoresis or by anion-exchange HPLC, as described in Pearson and Reanier, J. Chrom. 255:137-149 (1983).

The nucleic acids described here, or fragments thereof, can be used as hybridization probes for a cDNA library to isolate the corresponding full length cDNA and to isolate other cDNAs which have a high sequence similarity to the gene or similar biological activity. Probes of this type preferably have at least 30 bases and may contain, for example, 50 or more bases. The probe may also be used to identify a cDNA clone corresponding to a full length transcript and a genomic clone or clones that contain the complete gene, including regulatory and promotor regions, exons and introns. An example of such a screen includes isolating the coding region of the gene by using the known DNA sequence to synthesize an oligonucleotide probe. Labeled oligonucleotides having a sequence complementary to that of the nucleic acids of the present invention can be used to screen a library of human cDNA, genomic DNA or mRNA to determine which members of the library the probe hybridizes to.

The sequence of the cloned genes and synthetic oligonucleotides can be verified using the chemical degradation method of Maxam and Gilbert, Methods in Enzymology 65:499-560 (1980). The sequence can be confirmed after the assembly of the oligonucleotide fragments into the double-stranded DNA sequence using the method of Maxam and Gilbert, supra, or the chain termination method for sequencing double-stranded templates of Wallace et al., Gene 16:21-26 (1981). Southern blot hybridization techniques can be carried out according to Southern et al., J. Mol. Biol. 98:503 (1975).

### B. Cloning Methods for the Isolation of Nucleotide Sequences Encoding the Desired Proteins

In general, the nucleic acids encoding the subject proteins are cloned from DNA sequence libraries that are made to encode copy DNA (cDNA) or genomic DNA. The particular sequences can be located by hybridizing with an oligonucleotide probe, the sequence of which can be derived from the sequences provided herein, which provides a reference for PCR primers and defines suitable regions for isolating aging-associated specific probes. Alternatively, where the sequence is cloned into an expression library, the expressed recombinant protein can be detected immunologically with antisera or purified antibodies made against the aging-associated protein of interest.

To make the cDNA library, one should choose a source that is rich in mRNA. The mRNA can then be made into cDNA, ligated into a recombinant vector, and transfected into a recombinant host for propagation, screening and cloning. Methods for making and screening cDNA libraries are well known (see, e.g., Gubler and Hoffman, Gene 25:263-269 (1983); and Sambrook, supra).

For a genomic library, the DNA is extracted from the tissue and either mechanically sheared or enzymatically digested to yield fragments of preferably about 5-100 kb. The fragments are then separated by gradient centrifugation from undesired sizes and are constructed in bacteriophage lambda vectors. These vectors and phage are packaged in vitro, as described in Sambrook et al., supra. Recombinant phages are analyzed by plaque hybridization as described in Benton and Davis, Science 196:180-182 (1977). Colony hybridization is carried out as generally described in Grunstein et al., Proc. Natl. Acad. Sci. USA. 72:3961-3965 (1975).

An alternative method combines the use of synthetic oligonucleotide primers with polymerase extension on an mRNA or DNA template. This polymerase chain reaction (PCR) method amplifies the nucleic acids encoding the protein of interest directly from mRNA, cDNA, genomic libraries or cDNA libraries. Restriction endonuclease sites can be incorporated into the primers. Polymerase chain reaction or other in vitro amplification methods may also be useful, for example, to clone nucleic acids encoding specific proteins and express said proteins, to synthesize nucleic acids that will be used as probes for detecting the presence of mRNA encoding aging-associated proteins in physiological samples, for nucleic acid sequencing, or for other purposes (see, U.S. Pat. Nos. 4,683,195 and 4,683,202). Genes amplified by a PCR reaction can be purified from agarose gels and cloned into an appropriate vector.

Appropriate primers and probes for identifying the genes encoding the aging-associated proteins from mammalian tissues are generated from comparisons of the sequences provided herein. For a general overview of PCR, see, Innis et al., PCR Protocols: A Guide to Methods and Applications, Academic Press, San Diego (1990).

Synthetic oligonucleotides can be used to construct genes. This is done using a series of overlapping oligonucleotides, usually 40-120 bp in length, representing both the sense and anti-sense strands of the gene. These DNA fragments are then annealed, ligated and cloned.

A gene involved in the onset of aging, for example, can be cloned using intermediate vectors before transformation into mammalian cells for expression. These intermediate vectors are typically prokaryote vectors or shuttle vectors. The proteins can be expressed in either prokaryotes, using standard methods well known to those of skill in the art, or eukaryotes as described infra.

### C. Expression in Eukaryotes

Standard eukaryotic transfection methods are used to produce eukaryotic cell lines, e.g., yeast, insect, or mammalian cell lines, which express large quantities of the aging-associated proteins which are then purified using standard techniques (see, e.g., Colley et al., J. Biol. Chem. 264:17619-17622 (1989); and Guide to Protein Purification, in Vol. 182 of Methods in Enzymology, Deutscher ed. (1990)).

Transformations of eukaryotic cells are performed according to standard techniques as described by Morrison, J. Bact., 132:349-351 (1977), or by Clark-Curtiss and Curtiss, Methods in Enzymology 101:347-362, R. Wu et al. (Eds) Academic Press, NY (1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, Sambrook et al., supra). It is only necessary that the particular genetic engineering procedure utilized be capable of successfully introducing at least one gene into the host cell which is capable of expressing the protein.

The particular eukaryotic expression vector used to transport the genetic information into the cell is not particularly critical. Any of the conventional vectors used for expression in eukaryotic cells may be used. Expression vectors containing regulatory elements from eukaryotic viruses are typically used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

The vectors usually include selectable markers which result in gene amplification, such as, e.g., thymidine kinase, aminoglycoside phosphotransferase, hygromycin B phosphotransferase, xanthine-guanine phosphoribosyl transferase, CAD (carbamyl phosphate synthetase, aspartate transcarbamylase, and dihydroorotase), adenosine deaminase, dihydrofolate reductase, asparagine synthetase and ouabain selection. Alternatively, high yield expression systems not involving gene amplification are also suitable, such as, e.g., using a baculovirus vector in insect cells, with a target protein encoding sequence under the direction of the polyhedrin promoter or other strong baculovirus promoters.

The expression vector of the present invention will typically contain both prokaryotic sequences that facilitate the cloning of the vector in bacteria as well as one or more eukaryotic transcription units that are expressed only in eukaryotic cells, such as mammalian cells. The vector may or may not comprise a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the transfected DNA integrates into the genome of the transfected cell, where the promoter directs expression of the desired gene. The expression vector is typically constructed from elements derived from different, well characterized viral or mammalian genes. For a general discussion of the expression of cloned genes in cultured mammalian cells, see, Sambrook et al., supra, Ch. 16.

The prokaryotic elements that are typically included in the mammalian expression vector include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells.

The expression vector contains a eukaryotic transcription unit or expression cassette that contains all the elements required for the expression of the senescence-associated protein encoding DNA in eukaryotic cells. A typical expression cassette contains a promoter operably linked to the DNA sequence encoding the senescence-associated protein and signals required for efficient polyadenylation of the transcript. The DNA sequence encoding the protein may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus, the long term repeat from various retroviruses such as murine leukemia virus, murine or Rous sarcoma virus and HIV (see, Enhancers and Eukaryotic Expression, Cold Spring Harbor Pres, Cold Spring Harbor, N.Y. (1983)).

In the construction of the expression cassette, the promoter is preferably positioned at about the same distance from the heterologous transcription start site as it is from the transcription start site in its natural setting. As is known in the art, some variation in this distance can, however, be accommodated without loss of promoter function.

In addition to a promoter sequence, the expression cassette should also contain a transcription termination region downstream of the structural gene to provide for efficient termination. The termination region may be obtained from the same gene as the promoter sequence or may be obtained from a different gene.

If the mRNA encoded by the structural gene is to be efficiently translated, polyadenylation sequences are also commonly added to the vector construct. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40, or a partial genomic copy of a gene already resident on the expression vector.

In addition to the elements already described, the expression vector of the present invention may typically contain other specialized elements intended to increase the level of expression of cloned genes or to facilitate the identification of cells that carry the transfected DNA. For instance, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The cDNAs encoding the proteins of the invention can be ligated to various expression vectors for use in transforming host cell cultures. The vectors typically contain gene sequences to initiate transcription and translation of the aging-associated gene of interest. These sequences need to be compatible with the selected host cell. In addition, the vectors preferably contain a marker to provide a phenotypic trait for selection of transformed host cells such as dihydrofolate reductase or metallothionein. Additionally, a vector might contain a replicative origin.

Cells of mammalian origin are illustrative of cell cultures useful for the production of, for example, the aging-associated protein. Mammalian cell systems often will be in the form of monolayers of cells although mammalian cell suspensions may also be used. Illustrative examples of mammalian cell lines include VERO and HeLa cells, Chinese hamster ovary (CHO) cell lines, W138, BHK, COS-7 or MDCK cell lines. NIH 3T3 or COS cells are preferred.

As indicated above, the vector, e.g., a plasmid, which is used to transform the host cell, preferably contains DNA sequences to initiate transcription and sequences to control the translation of the aging-associated protein gene sequence. These sequences are referred to as expression control sequences. Illustrative expression control sequences are obtained from the SV-40 promoter (Berman et al., Science 222:524-527 (1983)), the CMV I.E. Promoter (Thomsen et al., Proc. Natl. Acad. Sci. 81:659-663 (1984)) or the metallothionein promoter (Brinster et al., Nature 296:39-42 (1982)). The cloning vector containing the expression control sequences is cleaved using restriction enzymes, adjusted in size as necessary or desirable and ligated with sequences encoding the aging-associated protein by means well known in the art.

When higher animal host cells are employed, polyadenylation or transcription terminator sequences from known mammalian genes need to be incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague et al., J. Virol. 45:773-781 (1983)).

Additionally, gene sequences to control replication in the host cell may be incorporated into the vector such as those found in bovine papilloma virus type-vectors (see, Saveria-Campo, “Bovine Papilloma virus DNA a Eukaryotic Cloning Vector” In: DNA Cloning Vol.II: a Practical Approach (Glover Ed.), IRL Press, Arlington, Va. pp. 213-238 (1985)).

The transformed cells are cultured by means well known in the art. For example, such means are published in Biochemical Methods in Cell Culture and Virology, Kuchler, Dowden, Hutchinson and Ross, Inc. (1977). The expressed protein is isolated from cells grown as suspensions or as monolayers. The latter are recovered by well known mechanical, chemical or enzymatic means.

### IX. PURIFICATION OF THE PROTEINS FOR USE WITH THE INVENTION

After expression, the proteins of the present invention can be purified to substantial purity by standard techniques, including selective precipitation with substances as ammonium sulfate, column chromatography, immunopurification methods, and other methods known to those of skill in the art (see, e.g., Scopes, Protein Purification: Principles and Practice, Springer-Verlag, NY (1982); U.S. Pat. No. 4,673,641; Ausubel et al., supra; and Sambrook et al., supra).

A number of conventional procedures can be employed when a recombinant protein is being purified. For example, proteins having established molecular adhesion properties can be reversibly fused to the subject protein. With the appropriate ligand, the aging-associated protein, for example, can be selectively adsorbed to a purification column and then freed from the column in a relatively pure form. The fused protein is then removed by enzymatic activity. Finally, aging-associated protein can be purified using immunoaffinity columns.

### A. Purification of Proteins from Recombinant Bacteria

When recombinant proteins are expressed by the transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells typically, but not limited to, by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al., and Sambrook et al., both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents which are capable of solubilizing aggregate-forming proteins, such as SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques.

Alternatively, it is possible to purify proteins from bacteria periplasm. Where the protein is exported into the periplasm of the bacteria, the periplasmic fraction of the bacteria can be isolated by cold osmotic shock in addition to other methods known to those of skill in the art (see, Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

### B. Standard Protein Separation Techniques For Purifying Proteins

1. Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

2. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

3. Column Chromatography

The proteins of interest can also be separated from other proteins on the basis of their size, net surface charge, hydrophobicity and affinity for ligands. In addition, antibodies raised against proteins can be conjugated to column matrices and the proteins immunopurified. All of these methods are well known in the art.

It will be apparent to one of skill that chromatographic techniques can be performed at any scale and using equipment from many different manufacturers (e.g., Pharmacia Biotech).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

Table 1 below indicates genes by identification in the “Gene Name” column that demonstrate change in expression with aging. “Image CloneID” refers to the IMAGE Consortium library clone identification number. “GenBank ID” indicates the accession number of the gene in the GenBank database. “LifeSpan Cluster ID” refers to the clone identification number in the LifeSpan collection of Clusters. “LifeSpan HAD Master ID” indicates the gene identification number in the LifeSpan High Density Arrays. Where a gene is indicated in the “Direction of Differential Expression during Human Aging” column as “down-regulated in multiple old tissues” it means that the expression of the subject gene is significantly decreased with aging (i.e., in tissues from older individuals, or in tissues undergoing senescence) vs. the corresponding normal young or tissues. Where a tissue is indicated in as “Up-regulated in multiple old tissues” it means that the gene is expressed at higher levels in tissues undergoing senescence or in tissues from older individuals vs. the corresponding tissues from young healthy individuals. For example, CD18 is expressed at significantly higher levels in tissues from young healthy individuals than in tissues from older individuals. Conversely, the CGI-27 protein is expressed at significantly higher levels in tissues undergoing senescence than in normal healthy tissues.

TABLE 1

| Image Clone | | | Direction Differential Expression during | Life Span | Life Span HDA |
|---|---|---|---|---|---|
| ID | Gene Name | GenBank ID | Human Aging | Cluster ID | Master ID |
| 381144 | 43 kDa inositol polyphosphate 5- | Z31695 | DOWN-Regulated In Multiple OLD Tissues | 113 | 4833 |
| | phosphatase. | | | | |
| 489497 | 52 kD subunit of transcription factor TFIIH. | Y07595 | DOWN-Regulated In Multiple OLD Tissues | 4801 | 5327 |
| 51694 | AP-4 Adaptor Complex Beta4 Subunit | AF092094 | DOWN-Regulated In Multiple OLD Tissues | 25091 | 1245 |
| | mRNA | | | | |
| 114303 | butyrophilia (BTF5) mRNA | U90552 | DOWN-Regulated In Multiple OLD Tissues | 16808 | 1883 |
| 28155 | c-fos | K00650 | DOWN-Regulated In Multiple OLD Tissues | 1127 | 667 |
| 146633 | CD18-tumor necrosis factor receptor 2 | L04270 | DOWN-Regulated In Multiple OLD Tissues | 2974 | 2566 |
| | related protein | | | | |
| 135913 | CD36 (clone 21) | M98399 | DOWN-Regulated In Multiple OLD Tissues | 467 | 2356 |
| 266854 | DNA-binding protein (mbp-1) | M32019 | DOWN-Regulated In Multiple OLD Tissues | 5255 | 7113 |
| 360076 | DR-nm23 mRNA | U29656 | DOWN-Regulated In Multiple OLD Tissues | 14744 | 4682 |
| 626342 | HFREP-1 mRNA | D14446 | DOWN-Regulated In Multiple OLD Tissues | 7749 | 6193 |
| 206703 | Ikaros/LyF-1 homolog (hlk-1) | U40462 | DOWN-Regulated In Multiple OLD Tissues | 2596 | 222 |
| 38481 | insulin-like growth factor binding protein 5 | M65062 | DOWN-Regulated In Multiple OLD Tissues | 2636 | 170 |
| | (IGFBP-5) | | | | |
| 183087 | interleukin 3 receptor (hIL-3Ra) | M74782 | DOWN-Regulated In Multiple OLD Tissues | 2735 | 3021 |
| 273460 | KIAA0086 | D42045 | DOWN-Regulated In Multiple OLD Tissues | 8533 | 3956 |
| 726291 | lamin B2 (LAMB2) | M94362 | DOWN-Regulated In Multiple OLD Tissues | 2882 | 6424 |
| 194984 | mitochondrial ATPase coupling factor 6 | M37104 | DOWN-Regulated In Multiple OLD Tissues | 551 | 3129 |
| | subunit (ATP5A) | | | | |
| 430075 | Mitogen-activated protein kinase-activated | AA236814 | DOWN-Regulated In Multiple OLD Tissues | 25607 | 5022 |
| | protein | | | | |
| 278431 | mRNA, exon 1,2,3,4, clone:RES4-24A. | AB000464 | DOWN-Regulated In Multiple OLD Tissues | 5334 | 4008 |
| 274405 | multi-specific organic anion tranporter-E | AF168791 | DOWN-Regulated In Multiple OLD Tissues | 129480 | 3970 |
| 155237 | multispanning membrane protein | U94831 | DOWN-Regulated In Multiple OLD Tissues | 17341 | 2755 |
| 489261 | NDP. | X65882 | DOWN-Regulated In Multiple OLD Tissues | 3460 | 5321 |
| 194428 | nonmuscle myosin heavy chain-B | M69181 | DOWN-Regulated In Multiple OLD Tissues | 3276 | 3116 |
| | (MYH10) | | | | |
| 113453 | ornithine decarboxylase (ODC1) | M16650 | DOWN-Regulated In Multiple OLD Tissues | 7916 | 1854 |
| 196151 | phorbolin I | U03891 | DOWN-Regulated In Multiple OLD Tissues | 3678 | 3156 |
| 267089 | Pig 10 (PIG10) | AF010314 | DOWN-Regulated In Multiple OLD Tissues | 6193 | 3861 |
| 128070 | Ras-GRF2 | AF023130 | DOWN-Regulated In Multiple OLD Tissues | 6648 | 2202 |
| 650252 | subtilisin-like protein (PACE4) | M80482 | DOWN-Regulated In Multiple OLD Tissues | 3613 | 6295 |
| 363226 | synaptophysin (p38). | X06389 | DOWN-Regulated In Multiple OLD Tissues | 4583 | 4786 |
| 32715 | thimet oligopeptidase (metalloproteinase). | Z50115 | DOWN-Regulated In Multiple OLD Tissues | 4678 | 164 |
| 128056 | transcobalamin II | L02648 | DOWN-Regulated In Multiple OLD Tissues | 4757 | 2201 |
| 114288 | transducins (beta) like 1 | Y12781 | DOWN-Regulated In Multiple OLD Tissues | 17567 | 1882 |
| 362254 | transmembrane protein rnp24. | X92098 | DOWN-Regulated In Multiple OLD Tissues | 4870 | 4754 |
| 161595 | zyxin. | X95735 | DOWN-Regulated In Multiple OLD Tissues | 5309 | 2847 |
| 547009 | (AF1q) mRNA | U16954 | DOWN-Regulated In Multiple OLD Tissues | 319 | 5816 |
| 140113 | (hMAD-2) | U68018 | DOWN-Regulated In Multiple OLD Tissues | 57079 | 2448 |
| 172356 | (hnRNP) C | M16342 | DOWN-Regulated In Multiple OLD Tissues | 8335 | 6979 |
| 190822 | ADDUCIN GAMMA SUBUNIT | D67031 | DOWN-Regulated In Multiple OLD Tissues | 8734 | 3076 |
| 344141 | ADENYLATE CYCLASE, TYPE I | L05500 | DOWN-Regulated In Multiple OLD Tissues | 280 | 308 |
| 158868 | ADP-ribosylation factor-like protein 2 | L13687 | DOWN-Regulated In Multiple OLD Tissues | 304 | 2811 |
| | (ARL2) | | | | |
| 42284 | alanyl-tRNA synthetase | D32050 | DOWN-Regulated In Multiple OLD Tissues | 327 | 1012 |
| 270521 | alternative splicing factor mRNA | M72709 | DOWN-Regulated In Multiple OLD Tissues | 3819 | 3923 |
| 191832 | apolipoprotein | X04506 | DOWN-Regulated In Multiple OLD Tissues | 485 | 3086 |
| 27571 | beta adaptin 1 | M34175 | DOWN-Regulated In Multiple OLD Tissues | 644 | 655 |
| 327684 | cadherin-15 | D83542 | DOWN-Regulated In Multiple OLD Tissues | 3228 | 4593 |
| 179373 | CDC25Hu2═cdc25 | S78187 | DOWN-Regulated In Multiple OLD Tissues | 2990 | 6998 |
| 158870 | cellular retinoic acid-binding protein II | M68867 | DOWN-Regulated In Multiple OLD Tissues | 4236 | 2812 |
| | (CRABP) | | | | |
| 150267 | chorionic gonadotropin. | V00518 | DOWN-Regulated In Multiple OLD Tissues | 1913 | 6891 |
| 382034 | cone-specific cGMP phosphodiesterase | D45399 | DOWN-Regulated In Multiple OLD Tissues | 4213 | 4840 |
| | gamma | | | | |
| 685038 | diacylglycerol kinase zeta mRNA | U94905 | DOWN-Regulated In Multiple OLD Tissues | 1294 | 7586 |
| 279118 | DNA MISMATCH REPAIR PROTEIN | U03911 | DOWN-Regulated In Multiple OLD Tissues | 1332 | 7658 |
| | MSH2 | | | | |
| 41375 | DRES9 | X98654 | DOWN-Regulated In Multiple OLD Tissues | 35081 | 173 |
| 28141 | Duo | U94190 | DOWN-Regulated In Multiple OLD Tissues | 19095 | 662 |
| 362177 | endothelin receptor (ETR) | D90402 | DOWN-Regulated In Multiple OLD Tissues | 1486 | 4750 |
| 292390 | fumarylacetoacetate hydrolase | M55150 | DOWN-Regulated In Multiple OLD Tissues | 1713 | 4171 |
| 23353 | GAMMA-AMINOBUTYRIC-ACID | R38700 | DOWN-Regulated In Multiple OLD Tissues | 1781 | 537 |
| | RECEPTOR ALPHA-6 SUBUNIT | | | | |
| 32644 | glucose-6-phosphate dehydrogenase | X03674 | DOWN-Regulated In Multiple OLD Tissues | 1846 | 163 |
| | (G6PD). | | | | |
| 361807 | GNAT1 mRNA for transducin alpha-chain. | X15088 | DOWN-Regulated In Multiple OLD Tissues | 1997 | 4736 |
| 109166 | guanosine 5′-monophosphate synthase | U10860 | DOWN-Regulated In Multiple OLD Tissues | 1921 | 1741 |
| 269933 | Has2 | U54804 | DOWN-Regulated In Multiple OLD Tissues | 2021 | 275 |
| 246429 | heterogeneous nuclear ribonucleoprotein | S74678 | DOWN-Regulated In Multiple OLD Tissues | 2088 | 3604 |
| | complex K | | | | |
| 172237 | homologue of Synaptocanalin 1 | H41572 | DOWN-Regulated In Multiple OLD Tissues | 25933 | 6977 |
| 36371 | human homologue of Rattus norvegicus | AB027520 | DOWN-Regulated In Multiple OLD Tissues | 138994 | 874 |
| | TAPL mRNA for TAP-like ABC | | | | |
| | transporter | | | | |
| 159987 | human homologue of Mus Musculus | U20238 | DOWN-Regulated In Multiple OLD Tissues | 45432 | 2828 |
| | GTPase-Activating Protein GAPIII | | | | |
| 486658 | inositol 1,4,5-trisphosphate 3-kinase | D38169 | DOWN-Regulated In Multiple OLD Tissues | 8501 | 5228 |
| | isoenzyme. | | | | |
| 773110 | insulin receptor substrate-1 | S62539 | DOWN-Regulated In Multiple OLD Tissues | 2629 | 6606 |
| 360490 | Integral membrane protein dgcr2/idd | D79985 | DOWN-Regulated In Multiple OLD Tissues | 2644 | 4700 |
| 470517 | keratin type II(58 kD) | M21389 | DOWN-Regulated In Multiple OLD Tissues | 152118 | 5102 |
| 282249 | KIAA0080; homologue of synaptotagmin | D38522 | DOWN-Regulated In Multiple OLD Tissues | 8518 | 4074 |
| | XI | | | | |
| 40394 | K1AA0349 | AB002347 | DOWN-Regulated In Multiple OLD Tissues | 5429 | 6822 |
| 490998 | kinase A anchor protein. | X97335 | DOWN-Regulated In Multiple OLD Tissues | 2849 | 7412 |
| 67014 | Krueppel-related zinc finger protein (H-plk) | M55422 | DOWN-Regulated In Multiple OLD Tissues | 5198 | 1714 |
| 488435 | LIM protein (LPP) | U49957 | DOWN-Regulated In Multiple OLD Tissues | 2937 | 5284 |
| 125723 | Macrophage inflammatory protein 1 | D00044 | DOWN-Regulated In Multiple OLD Tissues | 3004 | 2147 |
| 743271 | metalloproteinase-2 inhibitor (TIMP-2) | J05593 | DOWN-Regulated In Multiple OLD Tissues | 3102 | 6536 |
| 47266 | MHC class I antigen-like glycoprotein | J04142 | DOWN-Regulated In Multiple OLD Tissues | 4621 | 177 |
| | (CD1D) | | | | |
| 29706 | microtubule-associated protein 1B | L06237 | DOWN-Regulated In Multiple OLD Tissues | 3152 | 721 |
| | (MAP1B) | | | | |
| 197657 | mitochondrial aldehyde dehydrogenase x | M63967 | DOWN-Regulated In Multiple OLD Tissues | 345 | 3173 |
| | gene | | | | |
| 82042 | mRNA for | X15183 | DOWN-Regulated In Multiple OLD Tissues | 2033 | 1639 |
| 35128 | mRNA for BAP2-alpha protein | AB015019 | DOWN-Regulated In Multiple OLD Tissues | 160581 | 846 |
| 626385 | N-acetylglucosamine-phosphate mutase | AF102265 | DOWN-Regulated In Multiple OLD Tissues | 18293 | 6194 |
| | mRNA | | | | |
| 36809 | neural cell adhesion molecule (CALL) | AF002246 | DOWN-Regulated In Multiple OLD Tissues | 3357 | 886 |
| 149394 | osf-2 mRNA for osteoblast specific factor 2 | D13666 | DOWN-Regulated In Multiple OLD Tissues | 3568 | 2606 |
| | (OSF-2os) | | | | |
| 338700 | pancreatic elastase IIA | M16652 | DOWN-Regulated In Multiple OLD Tissues | 151929 | 4623 |
| 325897 | prothymosin alpha | M14630 | DOWN-Regulated In Multiple OLD Tissues | 4055 | 4587 |
| 28140 | Pyst 1 | X93921 | DOWN-Regulated In Multiple OLD Tissues | 1423 | 661 |
| 190902 | receptor tyrosine kinase (HEK) | M83941 | DOWN-Regulated In Multiple OLD Tissues | 1508 | 3078 |
| 545088 | receptor-type protein tyrosine phosphasase | L09247 | DOWN-Regulated In Multiple OLD Tissues | 4038 | 5752 |
| | gamma (PTPRG) | | | | |
| 257626 | retinoblastoma susceptibility mRNA | M15400 | DOWN-Regulated In Multiple OLD Tissues | 4225 | 7654 |
| 111520 | Serum paraoxonase/arylesterase 1 | D84371 | DOWN-Regulated In Multiple OLD Tissues | 4378 | 1815 |
| 159103 | short chain acyl-CoA dehydrogenase | M26393 | DOWN-Regulated In Multiple OLD Tissues | 260 | 2816 |
| 166044 | sigma 3B protein. | X99459 | DOWN-Regulated In Multiple OLD Tissues | 74204 | 2878 |
| 487900 | sodium/glucose cotransporter-like protein | M95549 | DOWN-Regulated In Multiple OLD Tissues | 4464 | 5262 |
| | mRNA | | | | |
| 130721 | Spermidine/spermine N1-acetyltransferase | M77693 | DOWN-Regulated In Multiple OLD Tissues | 1295 | 2268 |
| 381080 | TAFII32 | U21858 | DOWN-Regulated In Multiple OLD Tissues | 4821 | 4832 |
| 297795 | tetranectin. | X64559 | DOWN-Regulated In Multiple OLD Tissues | 4669 | 4241 |
| 700584 | thiopurine methyltransferase | S62904 | DOWN-Regulated In Multiple OLD Tissues | 4679 | 6363 |
| 41922 | threonyl-tRNA synthetase | M63180 | DOWN-Regulated In Multiple OLD Tissues | 4686 | 1007 |
| 321678 | tissue-type plasminogen activator (t-PA) | M15518 | DOWN-Regulated In Multiple OLD Tissues | 17669 | 4471 |
| 209655 | transforming growth factor-beta type III | L07594 | DOWN-Regulated In Multiple OLD Tissues | 4676 | 3318 |
| | receptor (TGF-beta) | | | | |
| 328401 | Transitional endoplasmic reticulum ATPase | G23173 | DOWN-Regulated In Multiple OLD Tissues | 4856 | 4603 |
| 207912 | type I 5′ iodothyronine deiodinase | S48220 | DOWN-Regulated In Multiple OLD Tissues | 4937 | 3297 |
| 290979 | tyrosine kinase mRNA | U02680 | DOWN-Regulated In Multiple OLD Tissues | 13817 | 7169 |
| 267022 | ubiquitin-conjugating enzyme UbcH7. | AJ000519 | DOWN-Regulated In Multiple OLD Tissues | 5006 | 3859 |
| 24781 | Unknown Gene | | DOWN-Regulated In Multiple OLD Tissues | | 573 |
| 773422 | Unknown Gene | | DOWN-Regulated In Multiple OLD Tissues | | 6619 |
| 177856 | Unknown Gene | | DOWN-Regulated In Multiple OLD Tissues | | 6994 |
| 186205 | Unknown Gene | | DOWN-Regulated In Multiple OLD Tissues | | 3039 |
| 428541 | Unknown Gene | | DOWN-Regulated In Multiple OLD Tissues | | 4963 |
| 172326 | Unknown Gene | | DOWN-Regulated In Multiple OLD Tissues | | 6978 |
| 647112 | Unknown Gene | | DOWN-Regulated In Multiple OLD Tissues | | 6264 |
| 113943 | Unknown Gene | | DOWN-Regulated In Multiple OLD Tissues | | 1870 |
| 364111 | Unknown Gene | | DOWN-Regulated In Multiple OLD Tissues | | 326 |
| 489983 | Unknown Gene | | DOWN-Regulated In Multiple OLD Tissues | | 7406 |
| 306032 | Unknown Gene | | DOWN-Regulated In Multiple OLD Tissues | | 7193 |
| 197077 | Unknown Gene | | DOWN-Regulated In Multiple OLD Tissues | | 3166 |
| 28308 | Unknown Gene | | DOWN-Regulated In Multiple OLD Tissues | | 161 |
| 142969 | Unknown Gene | | DOWN-Regulated In Multiple OLD Tissues | | 6878 |
| 428960 | Unknown Gene | | DOWN-Regulated In Multiple OLD Tissues | | 4975 |
| 178543 | Unknown Gene | | DOWN-Regulated In Multiple OLD Tissues | | 209 |
| 504351 | Unknown Gene | | DOWN-Regulated In Multiple OLD Tissues | | 5441 |
| 153377 | Unknown Gene | | DOWN-Regulated In Multiple OLD Tissues | | 6896 |
| 252400 | Unknown Gene | | DOWN-Regulated In Multiple OLD Tissues | | 3659 |
| 323396 | Unknown Gene | | DOWN-Regulated In Multiple OLD Tissues | | 7222 |
| 131132 | Unknown Gene | | DOWN-Regulated In Multiple OLD Tissues | | 2277 |
| 293133 | Unknown Gene | | DOWN-Regulated In Multiple OLD Tissues | | 4179 |
| 151231 | Unknown Gene | | DOWN-Regulated In Multiple OLD Tissues | | 2640 |
| 183613 | Unknown Gene | | DOWN-Regulated In Multiple OLD Tissues | | 3028 |
| 360838 | Unknown Gene | | DOWN-Regulated In Multiple OLD Tissues | | 4711 |
| 172477 | Unknown Gene | | DOWN-Regulated In Multiple OLD Tissues | | 2907 |
| 82627 | Unknown Gene | | DOWN-Regulated In Multiple OLD Tissues | | 1647 |
| 38578 | Unknown Gene | | DOWN-Regulated In Multiple OLD Tissues | | 922 |
| 20082 | Unknown Gene | | DOWN-Regulated In Multiple OLD Tissues | | 800 |
| 131799 | Unknown Gene | | DOWN-Regulated In Multiple OLD Tissues | | 6848 |
| 530813 | Unknown Gene | | DOWN-Regulated In Multiple OLD Tissues | | 404 |
| 382093 | Unknown Gene | | DOWN-Regulated In Multiple OLD Tissues | | 4842 |
| 325674 | Unknown Gene | | DOWN-Regulated In Multiple OLD Tissues | | 7239 |
| 280244 | Unknown Gene | | DOWN-Regulated In Multiple OLD Tissues | | 4035 |
| 243024 | Unknown Gene | | DOWN-Regulated In Multiple OLD Tissues | | 7067 |
| 51186 | Unknown Gene | | DOWN-Regulated In Multiple OLD Tissues | | 6957 |
| 115019 | Unknown Gene | | DOWN-Regulated In Multiple OLD Tissues | | 1911 |
| 755266 | Unknown Gene | | DOWN-Regulated In Multiple OLD Tissues | | 6548 |
| 503722 | Unknown Gene | | DOWN-Regulated In Multiple OLD Tissues | | 5419 |
| 41388 | Unknown Gene | | DOWN-Regulated In Multiple OLD Tissues | | 974 |
| 320839 | Unknown Gene | | DOWN-Regulated In Multiple OLD Tissues | | 7216 |
| 174234 | Unknown Gene | | DOWN-Regulated In Multiple OLD Tissues | | 6988 |
| 230408 | Unknown Gene | | DOWN-Regulated In Muitiple OLD Tissues | | 3459 |
| 183487 | Unknown Gene | | DOWN-Regulated In Multiple OLD Tissues | | 7007 |
| 364424 | Unknown Gene | | DOWN-Regulated In Multiple OLD Tissues | | 7324 |
| 40965 | Unknown Gene | | DOWN-Regulated In Multiple OLD Tissues | | 966 |
| 147318 | Unknown Gene | | DOWN-Regulated In Multiple OLD Tissues | | 2578 |
| 120291 | Unknown Gene | | DOWN-Regulated In Multiple OLD Tissues | | 2012 |
| 136363 | ZNFI31 | U09410 | DOWN-Regulated In Multiple OLD Tissues | 5207 | 2364 |
| 123066 | alpha-N-acetylgalactosaminidase | M62783 | Up-Regulated In Multiple OLD Tissues | 405 | 2069 |
| 33794 | ATP synthase gamma-subunit (L-type) | D16562 | Up-Regulated In Multiple OLD Tissues | 556 | 804 |
| 74314 | BiP/GRP | X87949 | Up-Regulated In Multiple OLD Tissues | 214 | 1463 |
| 79130 | cellular ligand of annexin II (p11) | M38591 | Up-Regulated In Multiple OLD Tissues | 8340 | 1557 |
| 193675 | CGI-27 protein | N66609 | Up-Regulated In Multiple OLD Tissues | 22511 | 3100 |
| 39874 | claudin-10 (CLDN10) | U89916 | Up-Regulated In Multiple OLD Tissues | 16800 | 941 |
| 115279 | COX17 | L77701 | Up-Regulated In Multiple OLD Tissues | 1176 | 1917 |
| 32577 | DNA polymerase gamma, mitochondrial | X98093 | Up-Regulated In Multiple OLD Tissues | 1343 | 7623 |
| | protein. | | | | |
| 726727 | ELL | U16282 | Up-Regulated In Multiple OLD Tissues | 1467 | 6430 |
| 430083 | erythroid membrane protein 4.1 | M61733 | Up-Regulated In Multiple OLD Tissues | 11147 | 5023 |
| 548663 | fau | X65923 | Up-Regulated In Multiple OLD Tissues | 102 | 7476 |
| 529085 | flavin-containing monooxygenase 1 | U87456 | Up-Regulated In Multiple OLD Tissues | 56910 | 5620 |
| | (FMO1) | | | | |
| 324127 | G protein beta subunit mRNA | AF195883 | Up-Regulated In Multiple OLD Tissues | 25360 | 4556 |
| 41629 | hevin like protein. | X86693 | Up-Regulated In Multiple OLD Tissues | 33121 | 986 |
| 204761 | histone H3.1 (H1F3) | M60746 | Up-Regulated In Multiple OLD Tissues | 2148 | 3260 |
| 545311 | HYA22 | D88153 | Up-Regulated In Multiple OLD Tissues | 57012 | 5764 |
| 595421 | Int-6 | U62962 | Up-Regulated In Multiple OLD Tissues | 15550 | 6115 |
| 625847 | KIAA0067 | D31891 | Up-Regulated In Multiple OLD Tissues | 2828 | 6188 |
| 358134 | L21 ribosomal protein | L38826 | Up-Regulated In Multiple OLD Tissues | 173 | 4670 |
| 510101 | lactate dehydrogenase B (LDH-B). | Y00711 | Up-Regulated In Multiple OLD Tissues | 2862 | 5489 |
| 589981 | lipocortin II | D00017 | Up-Regulated In Multiple OLD Tissues | 8317 | 6033 |
| 42674 | mCAF1 protein | U21855 | Up-Regulated In Multiple OLD Tissues | 21583 | 1020 |
| 545077 | mitochondrial succinate-ubiquinone | D10245 | Up-Regulated In Multiple OLD Tissues | 4555 | 5750 |
| | oxidoreductase iron sulfur subunit. | | | | |
| 153848 | mitochondrial ubiquinone-binding protein | M22348 | Up-Regulated In Multiple OLD Tissues | 4981 | 2707 |
| 78262 | mRNA for Hakata antigen | D88587 | Up-Regulated In Multiple OLD Tissues | 19417 | 1538 |
| 510658 | neutral amino acid transporter B | U53347 | Up-Regulated In Multiple OLD Tissues | 3418 | 5519 |
| 112621 | proliferation-associated gene (pag). | X67951 | Up-Regulated In Multiple OLD Tissues | 4683 | 1844 |
| 120804 | PROS-27 | X59417 | Up-Regulated In Multiple OLD Tissues | 3951 | 2022 |
| 156204 | proteinase activated receptor-2 | AA456265 | Up-Regulated In Multiple OLD Tissues | 98842 | 2776 |
| 75630 | ribosomal protein L11. | X79234 | Up-Regulated In Multiple OLD Tissues | 163 | 1486 |
| 204639 | ribosomal protein L23a | U37230 | Up-Regulated In Multiple OLD Tissues | 176 | 3256 |
| 252380 | ribosomal protein L27 (RPL27) | L19527 | Up-Regulated In Multiple OLD Tissues | 179 | 3658 |
| 179175 | ribosomal protein L32. | X03342 | Up-Regulated In Multiple OLD Tissues | 189494 | 2994 |
| 109355 | ribosomal protein L38. | Z26876 | Up-Regulated In Multiple OLD Tissues | 193 | 1749 |
| 74860 | ribosomal protein S10 | U14972 | Up-Regulated In Multiple OLD Tissues | 79 | 1475 |
| 238695 | ribosomal protein s3. | X55715 | Up-Regulated In Multiple OLD Tissues | 101 | 3524 |
| 418278 | RNA polymerase II larg subunit. | X63564 | Up-Regulated In Multiple OLD Tissues | 1383 | 4928 |
| 544875 | TATA binding protein-associated | M97388 | Up-Regulated In Multiple OLD Tissues | 4655 | 5735 |
| | phosphoprotein (DR1) | | | | |
| 150721 | TGIF protein. | X89750 | Up-Regulated In Multiple OLD Tissues | 122 | 2631 |
| 592243 | transcobalamin I | J05068 | Up-Regulated In Multiple OLD Tissues | 4756 | 6059 |
| 469526 | Translation repressor nat1 | U73824 | Up-Regulated In Multiple OLD Tissues | 4864 | 5051 |
| 664795 | UMP synthase | J03626 | Up-Regulated In Multiple OLD Tissues | 3564 | 6308 |
| 37402 | XP-C repair complementing protein | D21090 | Up-Regulated In Multiple OLD Tissues | 5087 | 7625 |
| | (p58/HHR23B) | | | | |
| 162345 | 20-kDa myosin light chain (MLC-2) | J02854 | Up-Regulated In Multiple OLD Tissues | 3290 | 2854 |
| 253061 | 5,6-dihydroxyindole-2-carboxylic acid | X51420 | Up-Regulated In Multiple OLD Tissues | 123 | 3676 |
| | oxidaae | | | | |
| 530942 | acetoacetyl-coenzyme A thiolase (EC | D90228 | Up-Regulated In Multiple OLD Tissues | 226 | 5674 |
| | 2.3.1.9). | | | | |
| 767779 | ACTIVATOR OF APOPTOSIS | D83699 | Up-Regulated In Multiple OLD Tissues | 254 | 6588 |
| | HARAKIRI | | | | |
| 34660 | adenovirus protein E3-14.7 k interacting | U41654 | Up-Regulated In Multiple OLD Tissues | 15211 | 825 |
| | protein 1 (FIP-1) mRNA, complete cds. | | | | |
| 27665 | adenylosuccinate lyase. | X65867 | Up-Regulated In Multiple OLD Tissues | 286 | 656 |
| 338490 | AFG3-like protein. | AJ001495 | Up-Regulated In Multiple OLD Tissues | 7589 | 4618 |
| 247235 | ANTISENSE BASIC FIBROBLAST | N57937 | Up-Regulated In Multiple OLD Tissues | 29167 | 3609 |
| | GROWTH FACTOR GFG | | | | |
| 72869 | beta nerve growth factor. | X52599 | Up-Regulated In Multiple OLD Tissues | 663 | 1419 |
| 377348 | calmodulin-like gene (CLP gene) | X13461 | Up-Regulated In Multiple OLD Tissues | 152264 | 4818 |
| 629159 | cardiac ventricular myosin light chain-2. | X66141 | Up-Regulated In Multiple OLD Tissues | 856 | 6234 |
| 28098 | clones 23667 and 23775 zinc finger protein | U90919 | Up-Regulated In Multiple OLD Tissues | 16856 | 668 |
| 275028 | cystathionine-beta-synthase | L00972 | Up-Regulated In Multiple OLD Tissues | 1151 | 3974 |
| 201919 | cystatin B | L03558 | Up-Regulated In Multiple OLD Tissues | 1154 | 3214 |
| 47795 | DBI | D28118 | Up-Regulated In Multiple OLD Tissues | 4112 | 1161 |
| 296716 | DEAD-box protein p72 (P72) | U59321 | Up-Regulated In Multiple OLD Tissues | 15748 | 4226 |
| 503206 | DNA-binding protein (SMBP2) mRNA, | L14754 | Up-Regulated In Multiple OLD Tissues | 1373 | 7423 |
| | complete cds. | | | | |
| 50375 | elongation factor EF-1-alpha | J04617 | Up-Regulated In Multiple OLD Tissues | 1469 | 1216 |
| 136850 | elongation factor-1-beta. | X60489 | Up-Regulated In Multiple OLD Tissues | 1470 | 2376 |
| 725232 | ets domain protein ERF | U15655 | Up-Regulated In Multiple OLD Tissues | 1567 | 442 |
| 510635 | fatty acid binding protein (FABP) | M10050 | Up-Regulated In Multiple OLD Tissues | 1627 | 5516 |
| 279308 | gamma-aminobutyric acidA receptor alpha | S62907 | Up-Regulated In Multiple OLD Tissues | 1777 | 4024 |
| | 2 subunit | | | | |
| 60874 | GATA2 | M68891 | Up-Regulated In Multiple OLD Tissues | 1485 | 7596 |
| 546398 | glutamate transporter MEAAC2 | U08989 | Up-Regulated In Multiple OLD Tissues | 1592 | 5790 |
| 21826 | glutamine synthase. | X59834 | Up-Regulated In Multiple OLD Tissues | 1866 | 483 |
| 74021 | Glycine amidinotransferase | D82580 | Up-Regulated In Multiple OLD Tissues | 1893 | 1455 |
| 545038 | guanylate binding protein isoform II (GBP- | M55543 | Up-Regulated In Multiple OLD Tissues | 2700 | 5746 |
| | 2) mRNA, complete cds. | | | | |
| 290091 | guanylate cyclase mRNA | L13436 | Up-Regulated In Multiple OLD Tissues | 576 | 4135 |
| 68330 | hCDC10 | S72008 | Up-Regulated In Multiple OLD Tissues | 923 | 1315 |
| 416914 | Heat shock 27 kd protein | Z23090 | Up-Regulated In Multiple OLD Tissues | 162 | 4895 |
| 127118 | hnRNP-E1 mRNA | Z29505 | Up-Regulated In Multiple OLD Tissues | 14614 | 2177 |
| 251319 | homologue of Mus musculus putative | G27595 | Up-Regulated In Multiple OLD Tissues | 897 | 3654 |
| | CCAAT binding factor 1 (mCBF) | | | | |
| 724379 | Human MHC class I transplantation antigen | J00191 | Up-Regulated In Multiple OLD Tissues | 11515 | 6396 |
| | (hla) gene. | | | | |
| 129059 | interleukin-7 receptor (IL-7) | M29696 | Up-Regulated In Multiple OLD Tissues | 2744 | 2224 |
| 119955 | KIAA 1226, homologous to Sus scrofa | D11336 | Up-Regulated In Multiple OLD Tissues | 23015 | 1996 |
| | mRNA for soluble angiotesin-binding | | | | |
| | protein. | | | | |
| 489069 | KIAA0038 | D26068 | Up-Regulated In Multiple OLD Tissues | 2822 | 5316 |
| 418130 | KIAA0076 | D38548 | Up-Regulated In Multiple OLD Tissues | 2392 | 4922 |
| 486530 | KIAA0102 | D14658 | Up-Regulated In Multiple OLD Tissues | 3548 | 5219 |
| 746193 | KIAA0148 | D63482 | Up-Regulated In Multiple OLD Tissues | 2410 | 445 |
| 223214 | KIAA0160 | D63881 | Up-Regulated In Multiple OLD Tissues | 8715 | 3430 |
| 297058 | L-PLASTIN | J02923 | Up-Regulated In Multiple OLD Tissues | 2868 | 4231 |
| 489103 | Lysosome membrane protein II | D12676 | Up-Regulated In Multiple OLD Tissues | 2983 | 5317 |
| 650222 | M2-type pyruvate kinase | M23725 | Up-Regulated In Multiple OLD Tissues | 4125 | 7568 |
| 38998 | Mad4 homolog (Mad4) | AF040963 | Up-Regulated In Multiple OLD Tissues | 7235 | 933 |
| 429667 | manganese superoxide dismutase (EC | X07834 | Up-Regulated In Multiple OLD Tissues | 4566 | 5005 |
| | 1.15.1.1). | | | | |
| 545458 | MHC class I-related protein | L14848 | Up-Regulated In Multiple OLD Tissues | 3137 | 5769 |
| 487132 | MITOCHONDRIAL ATP SYNTHASE D- | AA081285 | Up-Regulated In Multiple OLD Tissues | 31956 | 5243 |
| | SUBUNIT | | | | |
| 178015 | mitogen- and stress-activated protein | H41647 | Up-Regulated In Multiple OLD Tissues | 26314 | 2978 |
| | kinase-2 (MSK2) | | | | |
| 239219 | MyD118 | AF090950 | Up-Regulated In Multiple OLD Tissues | 40414 | 3535 |
| 296134 | Nadh-ubiquinone oXIdoreductase chain 6 | X84075 | Up-Regulated In Multiple OLD Tissues | 7518 | 4216 |
| 34483 | NADH:ubiquinone oxidoreductase subunit | U53468 | Up-Regulated In Multiple OLD Tissues | 3320 | 818 |
| | B13 (B13) | | | | |
| 526223 | nascent-polypeptide-associated complex | X80909 | Up-Regulated In Multiple OLD Tissues | 33131 | 5593 |
| | alpha polypeptide (NACA) mRNA | | | | |
| 28332 | PDGFB | M12783 | Up-Regulated In Multiple OLD Tissues | 3777 | 684 |
| 645512 | PTD010 | W22147 | Up-Regulated In Multiple OLD Tissues | 17996 | 6258 |
| 274217 | putative tetraspan transmembrane protein | AF027204 | Up-Regulated In Multiple OLD Tissues | 6765 | 3965 |
| | L6H (TM4SF5) | | | | |
| 28021 | pyrroline 5-carboxylate reductase | M77836 | Up-Regulated In Multiple OLD Tissues | 4118 | 673 |
| 362136 | Rab12 | Z22818 | Up-Regulated In Multiple OLD Tissues | 31162 | 4748 |
| 741406 | rat interactor (RINI). | L36463 | Up-Regulated In Multiple OLD Tissues | 4147 | 6499 |
| 309449 | ribosomal protein (RPS4Y) | M58459 | Up-Regulated In Multiple OLD Tissues | 105 | 4421 |
| 470040 | ribosomal protein L30 | L05095 | Up-Regulated In Multiple OLD Tissues | 185 | 5080 |
| 430270 | ribosomal protein L30 mRNA | M94314 | Up-Regulated In Multiple OLD Tissues | 177 | 5028 |
| 590067 | ribosomal protein L31. | X15940 | Up-Regulated In Multiple OLD Tissues | 186 | 6034 |
| 544545 | ribosomal protein L35 mRNA | U12465 | Up-Regulated In Multiple OLD Tissues | 189 | 5717 |
| 125068 | Ribosomal protein L44 | U01925 | Up-Regulated In Multiple OLD Tissues | 197 | 6763 |
| 471253 | ribosomal protein S12. | X53505 | Up-Regulated In Multiple OLD Tissues | 81 | 5133 |
| 301797 | ribosomal protein S13 | L01124 | Up-Regulated In Multiple OLD Tissues | 82 | 4325 |
| 530065 | ribosomal protein S24 mRNA. | M31520 | Up-Regulated In Multiple OLD Tissues | 94 | 5636 |
| 759948 | S100 protein beta-subunit gene | M59488 | Up-Regulated In Multiple OLD Tissues | 33572 | 6583 |
| 628810 | sarcomeric mitochondrial creatine kinase | J05401 | Up-Regulated In Multiple OLD Tissues | 151906 | 7555 |
| | (MtCK) | | | | |
| 530814 | selenoprotein P. | Z11793 | Up-Regulated In Multiple OLD Tissues | 4327 | 5668 |
| 76385 | signal peptidase | G29980 | Up-Regulated In Multiple OLD Tissues | 19483 | 1501 |
| 300611 | Stratum Corneum Tryptic Enzyme (SCTE) | AF168768 | Up-Regulated In Multiple OLD Tissues | 29918 | 4296 |
| 531035 | TAX responsive element binding protein | X81987 | Up-Regulated In Multiple OLD Tissues | 56954 | 5680 |
| | 107. | | | | |
| 79272 | thymosin beta-10 | S54005 | Up-Regulated In Multiple OLD Tissues | 4706 | 1566 |
| 77085 | thyroid hormone receptor coactivating | AF016270 | Up-Regulated In Multiple OLD Tissues | 6424 | 1510 |
| | protein | | | | |
| 35326 | TNFSF4 (tumor necrosis factor | D90224 | Up-Regulated In Multiple OLD Tissues | 3579 | 843 |
| | superfamily, member 4) | | | | |
| 760220 | transcription factor TFIIE alpha. | X63468 | Up-Regulated In Multiple OLD Tissues | 4807 | 6586 |
| 503085 | TSC-22 related protein (TSC-22R) | AA147844 | Up-Regulated In Multiple OLD Tissues | 86463 | 5409 |
| 30394 | ubiquitin specific protease 9 | X98296 | Up-Regulated In Multiple OLD Tissues | 3884 | 731 |
| 527027 | Unknown Gene | | Up-Regulated In Multiple OLD Tissues | | 5612 |
| 757060 | Unknown Gene | | Up-Regulated In Multiple OLD Tissues | | 6563 |
| 291633 | Unknown Gene | | Up-Regulated In Multiple OLD Tissues | | 4160 |
| 629587 | Unknown Gene | | Up-Regulated In Multiple OLD Tissues | | 6238 |
| 182188 | Unknown Gene | | Up-Regulated In Multiple OLD Tissues | | 3012 |
| 277422 | Unknown Gene | | Up-Regulated In Multiple OLD Tissues | | 7142 |
| 725493 | Unknown Gene | | Up-Regulated In Multiple OLD Tissues | | 6413 |
| 322334 | Unknown Gene | | Up-Regulated In Multiple OLD Tissues | | 4491 |
| 362329 | Unknown Gene | | Up-Regulated In Multiple OLD Tissues | | 4756 |
| 238346 | Unknown Gene | | Up-Regulated In Multiple OLD Tissues | | 3512 |
| 611924 | Unknown Gene | | Up-Regulated In Multiple OLD Tissues | | 7539 |
| 75268 | Unknown Gene | | Up-Regulated In Multiple OLD Tissues | | 6691 |
| 530551 | Unknown Gene | | Up-Regulated In Multiple OLD Tissues | | 5656 |
| 22750 | Unknown Gene | | Up-Regulated In Multiple OLD Tissues | | 524 |
| 755035 | Unknown Gene | | Up-Regulated In Multiple OLD Tissues | | 6545 |
| 194484 | Unknown Gene | | Up-Regulated In Multiple OLD Tissues | | 3117 |
| 360931 | Unknown Gene | | Up-Regulated In Multiple OLD Tissues | | 4715 |
| 25530 | Unknown Gene | | Up-Regulated In Multiple OLD Tissues | | 595 |
| 563318 | Unknown Gene | | Up-Regulated In Multiple OLD Tissues | | 5921 |
| 531450 | Unknown Gene | | Up-Regulated In Multiple OLD Tissues | | 5692 |
| 471214 | Y box binding protein-1 (YB-1) mrNA. | J03827 | Up-Regulated In Multiple OLD Tissues | 73193 | 5131 |

What is claimed is:

1. A method for detecting whether a tissue is undergoing senescence, said method comprising the step of detecting the overexpression or the underexpression of a senescence-associated molecule of interest according to Table 1 in a subject, wherein overexpression or underexpression of said molecule is indicative of senescence.

2. The method of claim 1, wherein overexpression of said molecule is indicative of senescence, and wherein said molecule is overexpressed in said tissue.

3. The method of claim 1, wherein underexpression of said molecule is indicative of senescence, and wherein said molecule is underexpressed in said tissue.

4. The method of claim 1, said method comprising detecting an mRNA encoding said senescence-associated molecule.

5. The method of claim 1, said method comprising detecting said senescence-associated molecule in an immunoassay.

6. A method for identifying a modulator of senescence, said method comprising the steps of:
(a) culturing a cell in the presence of said modulator to form a first cell culture;
(b) contacting RNA or cDNA from said first cell culture with a probe which comprises a polynucleotide sequence that encodes a senescence-associated protein selected from the group consisting of the sequences set forth in Table 1;
(c) determining whether the amount of said probe which hybridizes to the RNA or cDNA from said first cell culture is increased or decreased relative to the amount of the probe which hybridizes to RNA or cDNA from a second cell culture grown in the absence of said modulator; and
(d) detecting the presence or absence of an increased proliferative potential in said first cell culture relative to said second cell culture.

7. The method of claim 6, wherein said first and second cell cultures are obtained from a fibroblast cell.

8. A method for identifying a modulator of a young cell, said method comprising the steps of:
(a) culturing the cell in the presence of the modulator to form a first cell culture;
(b) contacting RNA from the first cell culture with a probe which comprises a polynucleotide sequence associated with senescence, wherein the sequence is selected from the group consisting of sequences set out in Table 1;
(c) determining whether the amount of said probe which hybridizes to the RNA from said first cell culture is increased or decrease relative to the amount of said probe which hybridizes to RNA from a second cell culture grown in the absence of said modulator; and
(d) detecting the presence of an increased proliferative potential in the first cell culture relative to the second cell culture.

9. The method of claim 8, wherein said first and second cell cultures are obtained from a fibroblast cell.

10. A method for inhibiting cell senescence, said method comprising the step of introducing into a cell a senescence-associated molecule according to Table 1, wherein underexpression of said senescence-associated molecule is indicative of senescence.

11. The method of claim 10, wherein said senescence-associated molecule is a nucleic acid encoding a senescence-associated protein.

12. The method of claim 10, wherein said senescence-associated molecule is a protein.

13. A method for inhibiting cell senescence, said method comprising the step of inhibiting in a cell a senescence-associated molecule according to Table 1, wherein overexpression of said senescence-associated molecule is indicative of senescence.

14. The method of claim 13, wherein said senescence-associated molecule is inhibited using an antisense polynucleotide.

15. The method of claim 13, wherein said senescence-associated molecule is inhibited using an antibody that specifically binds to the senescence-associated protein.

16. A method for inhibiting cell senescence in a patient in need thereof, said method comprising the step of administering to the patient a compound that modulates the senescence of a cell.

17. A kit for detecting whether a cell is undergoing senescence, said kit comprising:
(a) a probe which comprises a polynucleotide sequence according to Table 1, associated with aging; and
(b) a label for detecting the presence of said probe.
